SEQUENCE LISTING (6 sequences)

SEQ ID NO: 1 - protein, 359 aa, Homo sapiens
METQLSNGPTCNNTANGPTTINNNCSSPVDSGNTEDSKTNLIVNYLPQNMTQEELKSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGISRGVGFIRFDKRIEAEEAIKGLNGQKPPGATEPITVKFANNPSQKTNQAILSQLYQSPNRRYPGPLAQQAQRFRLDNLLNMAYGVKRFSPMTIDGMTSLAGINIPGHPGTGWCIFVYNLAPDADESILWQMFGPFGAVTNVKVIRDFNTNKCKGFGFVTMTNYDEAAMAIASLNGYRLGDRVLQVSFKTNKTHKA

SEQ ID NO: 2 - protein, 367 aa, Homo sapiens
MVTQILGAMESQVGGGPAGPALPNGPLLGTNGATDDSKTNLIVNYLPQNMTQDEFKSLFGSIGDIESCKLVRDKITGQSLGYGFVNYSDPNDADKAINTLNGLKLQTKTIKVSYARPSSASIRDANLYVSGLPKTMSQKEMEQLFSQYGRIITSRILVDQVTGVSRGVGFIRFDKRIEAEEAIKGLNGQKPLGAAEPITVKFANNPSQKTGQALLTHLYQSSARRYAGPLHHQTQRFRLDNLLNMAYGVKSPLSLIARFSPIAIDGMSGLAGVGLSGGAAGAGWCIFVYNLSPEADESVLWQLFGPFGAVTNVKVIRDFTTNKCKGFGFVTMTNYDEAAMAIASLNGYRLGERVLQVSFKTSKQHKA

SEQ ID NO: 3 - protein, 366 aa, Homo sapiens
MVMIISTMEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGVSRGVGFIRFDKRIEAEEAIKGLNGQKPSGATEPITVKFANNPSQKSSQALLSQLYQSPNRRYPGPLHHQAQRFRLDNLLNMAYGVKRFSPITIDGMTSLVGMNIPGHTGTGWCIFVYNLSPDSDESVLWQLFGPFGAVNNVKVIRDFNTNKCKGFGFVTMTNYDEAAMAIASLNGYRLGDRVLQVSFKTNKAHKS

SEQ ID NO: 4 - RNA, 3983 nt, Homo sapiens
  source 1..3983: mol_type = mRNA
aagacgtcctcccccctatttctgttcttagttagagatgcagcagcttactcctgtagcgacctactaaaaagcaacaaggagaaagacatcgtctttttgaaaaaacgttctttcgtctcttttctttgttccttggtttgtttttcttgcccctttttgtttagttgaacggcaataggagggtagtctctccgtctttttaaactcttttttaagtttcccctcccctttcatattttttttcgccatttcttttagcattggactttggggtcgaaagcgtttctttttatttgcttcttttaagccgagcacagtttaggtttcgtgctgtcttaagagaactatccagcagcttcttgctcatccttattgggagaactgcaccgttactttaaaaacacacatacacaaaaaccttaagggagaaagcaggtaattgctgccatggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacagggcagagcttgggatatggctttgtgaactacattgaccccaaggatgcagagaaagctatcaacaccctgaatggattgagacttcaaaccaaaacaataaaagtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggttggacaatctgctcaatatggcttatggagtaaagaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatagctagcctcaatggataccgtctgggagacagagtactgcaggtctcctttaagacaaacaaaacgcacaaagcctaatgagctcttgtcctcagtccatttatatatgaaaactatacaacaaaggcaagttaagagaaactttatacattagtaaatgtctttgtaagtcagtgttgagatggggataaaatgactacttagcatcctaagaaatatgtgagattttttattgctagtatttgaattaaaacttcttaaatatcttttatgtttgaatatggacaagaggtacagggtttttacctgtcacattgcattctattgccttctttgaagaaggtggaccttttaaagtgtttcagctaagggaagacatttcttttctttttacataactgccttgaacctgtgagtaaatattgaggctttgtgttgtaattcttcagttggttgtgtcttttttttcccccctttttttcctttttctgattagctttgtgtttggtttacatttaaagcattgctgttatgtctgtttaagaaaagtattttgaagtttacatttttatttatgaagtttaaaacagtatttattttgtaattatgatttgggttggggaagggggggctacattataaacgcttattgtaagaatactggagaacttttcgtaaagcagtaccttgccaaagagataagagcctctttgatgtgggtttaaaaaaagcatctatttttataaaaaagaaaatttggagaaactttttactggtcctggaacaaatattttgacttgaatactttgagaaatctcttcatatgacacctagtgagcttttaaaatttaccaggaaatttgcagcggttggaaaatttagaaagatttatggtgtagaaaatacttttgagatctttgtatgaaaggagtagaatcaatggggggaaacactgctggtttcatttttgtaatcaccagtggagcgtctgatcatcctggttattatgtgataggtggctcacattgatttgtgattttgaaacaaataaaaaaaatttacaaaagaatatataagagcaggcaagaaatttaaattaccgagagatgggggaaaaaatctgttcttcctaaagaaatcccttcagatagagctcatggtgtttagtgatgtacttgcagtattgtttgaagaattgttttgtcttaaggaaaaaagacgttgcacatgatttgtactgcagcaaatcagcaaaagtgatctgagttggatatatttgaaggtattttgaaagttacgttcaaggctaacacctgagctttgtgtaatgtaaataagaccttgtgtttatgaacctttcagctaatttaattttttttcccttacatgccaagtgatgttcaggttttgaatgtttttgtatcagttttttcctttgtaaatggcattaacattgttacttgaggtcttgcttaatcacttttgttgtcctgaggacttgaatttacagtgcatcagatttgttgcaaattttgtctgtagatagtctagcttcagctgtttatggtgatgctacattttggtttataaatatgtttgtggtataaaaaaatgagtataaccataggttttgaacaaatttccttacatttttcatacaaaaatcataaatatctgtatgctattgaaatttaactttgtatgatgcttaaaaaccactatttggggaaataataaaataagtctttaccatgtatgaaagaaattttaaaaaatacaaaatattttctgattagcatctagcttataataaattttcaaaaaagctgaaggcaaaaatgccttcatcaggatgcactgagaactatatagttacgtcctgctttttgtataaactgagatgctcacatgcttccccttagaacaggcaatgtgctatgcataacatagttgtacattatctttgcggttgctttgagttttattttttattatttaaaattgtagttataaaatttttcagtatagtacagtacatatactgtgaggcgcgtgctaaagtgaataagcgagttttcatgctgacccactcaatgctattcagaaatcaattggcttagcactttctcatatccttaggtgcatttagattgccagagttaaccttctgcgtttaaaaaaagaaaaacactaaaaaataaaatacatgtatatacttaaaaaaaaataataaggtttccctcaagggaaaacagcagctacatgcttctttcctatactactgtagcaaaccaaggcattgatgagagggcatgcaaattgtgcttcactttacagtgttttatcagagcacttaataaaatgtaaggctggtatttatttgaagttgtacagtatgacttaattcacatctgttggaatagaaaatatattctgttgagtatttaagaggctgtacatgttttcttttgtgtttggattctttgtactttttcatgttcagtacatcaataaacaaagttgaagggaaa

SEQ ID NO: 5 - RNA, 4742 nt, Homo sapiens
  source 1..4742: mol_type = mRNA
agagcgcggagcgcgcagcgcgggccggagggagggagggagggtgggcgaggggagaggacttgaacacacgggaccggacaggaccgaccgaggggcggcggcgaaaggcagagcgccgcgatctctgtcgggaagcgcaacctccccgggccccgcggggccgcgcagggggcgtcctcaaccccgcgcccgctccctctttccatcccctgccgcccgcaggccaccccggggcccggccatccgcgcgcgcatccccgggttcgggcccgtccctggccctcgagggagccgccgccttcatcgccacatctgcagcggccgcaccagaggccgcccgggcgggaccccagcgtgagcatcgggcgcccccctaggagtgcaccacccccggagcccccctcaacacggaccgcgcccgccgggcacacaagaatggtcactcagatactgggggccatggagtctcaggtgggggggggcccggccggcccggccctgcccaacgggccactccttggtacaaatggagccactgacgacagcaagaccaacctcatcgtcaactacctgccccagaacatgacccaggatgagttcaagagtctcttcggcagcattggcgacatcgagtcctgcaagttggttcgggacaagatcacagggcagagccttggctacgggtttgtgaactattctgaccccaatgatgcagacaaagccatcaacaccctcaacggcctcaaattacagacgaagaccatcaaggtgtcctatgccagacccagttcagcatccatccgggatgctaacctgtacgtcagcgggctccccaagaccatgagccagaaagagatggagcagctcttctcccagtacggccgcatcatcacgtcccgcatcctggtggaccaggtcacaggtgtctctcggggtgtgggattcatccgctttgacaagaggattgaggccgaagaggctatcaaaggactgaatgggcagaagccgctgggcgcagctgagcccatcacagtcaagttcgcgaacaacccaagtcagaagacggggcaggcgctgctcacccacctctaccagtcatccgcccggcgctacgcaggccccctacaccatcagacccagcgtttccggctggacaatttgctcaacatggcctacggcgtcaagagtcccctgtcgctcatcgccaggttctcgccgatcgccatcgatggtatgagcggcctggcgggcgtgggcctgtcggggggcgcggcgggcgccggctggtgcatcttcgtgtacaacctgtcaccggaggcagacgagagcgtgctgtggcagctgttcgggccttttggggcagtcaccaacgtcaaggtcatccgtgatttcaccaccaacaagtgcaagggtttcggcttcgtgaccatgaccaactatgacgaggcggccatggccatcgccagcctgaacggctatcgcctgggcgagcgcgtgctgcaggtctccttcaagaccagcaaacagcacaaggcgtgagcccaccccgcctgccctcccaccccctccccgggcagcagagagagagagagagaaagagagagagagagagagaaggggcccaagagagacagcacaggcagccccacggacgacgcgagggccccacgtccctgcggaagccacagggtgagcactctggggtgggagggtctgcagggaattgggggggtgcccggggatcccccgccccatcctcctgcccccaccccaggctgggctgttcactctctcgtcttggtttggttcatggtgaaggtttttgtttcttttttcggctaaaaagaatgcagagatgtgcccccacccccaccctcgaccacccccgatgggatggcttggggggctccagggggtgccctcccagacccccttgcccaggcctccccagcacctaggtggggcctggggtaggaggaacaggtttaaaaatccccaaaaaagcgaaccgtgaggaggggtgtgggcacccccggcccagtgccccctggtggaatgcgggggagcaggcagtggggctggaagcagaaacaaaatgaaaaaaaaaggggggtgggaggggaagaaaaactctatttttgtaaaaagggaaaaagacctcgtggagaatttttactggggattcttgaacttgaaaaaaaaaaacacaaaaaaagacaaaaaaaaaaaaagaaaatattttggcaggttatgtttaccaactggggcgggggtggggggggcccagggagcagggcttaggggctagcagcccacggggccacacagagaaacaaccacgcagacagtcacaccacggggacacacggacagacgcagacggacacagcgacatgccccagacacgttaagggactggttggccaaactcagacacgtggacagggatagacagaaagagacagaagctggggctacgtccatgtggacacagaccacagatgtgggcacacgaatccatccacctgtccacgtgcacacgtgaatgtagcgatagatatttggacatcaaatttggacaccaggtcacggagaaacacaaacaccacccaggaacacgcagacatatgcccatagcatcccacaggcccaggcaggaggtcccaccccagaccctgccccagacgctccctcaccctctcggccccctcgccctggcccccaggttctctgcagagatctctcctggacccccagcggtctccttggcgcccacgaacacaggcgtgcacacgcagcgcacatgcattgagacacacgatacctcgttccaccttggcgtcacggtgggctggaggcagcccccccccccacaacacttgaggccaaaggacacccctgtccccggggctcagcctccccctgggccgaggccttggcccacacagctgagggagaggcccagccctgagcctcccccaccaggactgggcctccccggcagcaagaccccggcactccccgccaggcccagggtggggtggatgggcccaaaggcccagccccacactcctttcccgtccacttgtcacctacctatcccctttcggtttgttgggtttttcgtctttccagattgcagtggacacagcccccgatctcgagccccggcccccggcttctgtctggacattgcatcgccctagttttctttctttaaaaaaaaaaaattccaacagaagccacaggccggagcccctgggagccctcgcaccgctccccaccccactcagcggccccatcggaactgagaattgcaaaacccccgactttagctatagttaagttgccttcccccggtgtccccacgtttggtgtctggctatggcgcccccatctgactgtccagctctctctcccccacccctcccggtgttgttattaaacgtctgtggagcttctgctgcaaggaacaaaaaagaaaaaaatcaaaaaagcgacaaaaaaacacaaacagaagaggaaaaaaaagcaacaaaaaaagaaaacacacagaagagatttaaaaaaaaaaaaggaaaaaaaaaaaagacataaactggcaccagttaactttcttgtacttttttgctgaatttagcttcttgtagttttaacttattgctatgttaactatttattctcctcgttgccctgtaaggacatccgtgtatatttctgttacttcatccggtttgcaagttaaaggaacgacaatgttctctttgtttctttaagttttgccgagacatggttatgcctaatttatttataaaaggggaagtggaatcattaaagtaataataattattaataacagtaatggtagccgagtggcacgcgggggcgtgtgctctgcgggacagtccccacggccagcgacgtccaggtcaccaatgggattctttttgctcttgtcttgagaattttttcagtcctatttagctggtgaaatccctagcttgttcttgatacacgaacctatttattctcgtggttttaagtcctccctgccctctctcctctcccctgcagcagggcagggacccctctcccctgctgtctcttggggctctctcccctccgccctctgcattcgggaacacgcacgtccgcgtgggaagcttgcagcagggcgtccgagcaataagggctgggtttgtcccccaccctggggggggccaggctccagagagggggcacctccccacacaccccccccccaccgaggcctaggcccctgccacccccaagactgggaggggacttctttttctaaaacacaaaactcagcccagccaggccccctccctggaggccagcccctccccgcagggggccaggccggggctcccaggcgagggggacttggggcttccacgtccctggggggcaggggccaggccaggggggagggggctcagcccctccacccctcccttcatcctgttatttattcggaaggtttcaaatcacgacagagtccatgttggaggtgataaaaaactgtaaaaaaaaaaaaaaaaaaaaagacaaaacaaaagacaaaaaaaaaaaaaaaacccaacaagaacacttcgcgttgcgtttagactatttattaccgggatcacaggcctggccgcggtaacagacttttacatggaattgtttaattatttgtacttttcatgccagaaaataaaagttcagaatcttaa

SEQ ID NO: 6 - RNA, 3965 nt, Homo sapiens
  source 1..3965: mol_type = mRNA
gagccaatttcagcaaggctctgttcagttgttcttatctacatcctagaatcgggggtttcagctcactgctccttttcttttttttctttctctcccccgcccacccccccaaaaataattgatttgctttacaatcatccacactgtgttttgtggatctttaattatatataacaatagtagtcattttaaatatatattctgaaatctttgcaaattttaacagaagagtcgaagctctgcgagacccaatatttgccaataagaatggttatgataattagcaccatggagcctcaggtgtcaaatggtccgacatccaatacaagcaatggaccctccagcaacaacagaaactgtccttctcccatgcaaacaggggcaaccacagatgacagcaaaaccaacctcatcgtcaactatttaccccagaatatgacccaagaagaattcaggagtctcttcgggagcattggtgaaatagaatcctgcaaacttgtgagagacaaaattacaggacagagtttagggtatggatttgttaactatattgatccaaaggatgcagagaaagccatcaacactttaaatggactcagactccagaccaaaaccataaaggtctcatatgcccgtccgagctctgcctcaatcagggatgctaacctctatgttagcggccttcccaaaaccatgacccagaaggaactggagcaacttttctcgcaatacggccgtatcatcacctcacgaatcctggttgatcaagtcacaggagtgtccagaggggtgggattcatccgctttgataagaggattgaggcagaagaagccatcaaagggctgaatggccagaagcccagcggtgctacggaaccgattactgtgaagtttgccaacaaccccagccagaagtccagccaggccctgctctcccagctctaccagtcccccaaccggcgctacccaggtccacttcaccaccaggctcagaggttcaggctggacaatttgcttaatatggcctatggcgtaaagaggttctccccaattaccattgatggaatgacaagccttgtgggaatgaacatccctggtcacacaggaactgggtggtgcatctttgtctacaacctgtcccccgattccgatgagagtgtcctctggcagctctttggcccctttggagcagtgaacaacgtaaaggtgattcgtgacttcaacaccaacaagtgcaagggattcggctttgtcaccatgaccaactatgatgaggcggccatggccatcgccagcctcaacgggtaccgcctgggagacagagtgttgcaagtttcctttaaaaccaacaaagcccacaagtcctgaatttcccattcttacttactaaaatatatatagaaatatatacgaacaaaacacacgcgcgcacacacacacatacacgaaagagagagaaacaaacttttcaaggcttatattcaaccatggactttataagccagtgttgcctaagtattaaaacattggattatcctgaggtgtaccaggaaaggattttataatgcttagaaaaaaagaaaaaaaaaaaacaaaaaatacctttgatgcattgaatgttctttcatagctgtcgttgttgaatataatatacatagatagcgatgtgcagggatttttacccagccagctaactttactaccttccttgaaggtggatctttttaagatgaccattcgctcatttgaagaagaaaaacaaaaaacaaaaaaaataataaaaataaaacaatttttacaaagtaatgggattcaaagaaaggaaaaaaagatttttcttttttgtcaaaatatcgatccaatcagattggtaaaaacccccacacaaattaaagaggaataataaaaattgcaaaaataaaaaaaaacttttgcaaatttttttatttttccttctttcttttatatcatgtgaactaaaacagtcttctgttaggggatgggggcaagggggatacctgatgacattaacaatttaataacattaacattgttgccaaagaggtggtctctttgctgaaaatgggtttcaagaaaaatctatttttataaaatataaagaatttttacaagagaatctggatttgagaaaaaaatattttgactggctaatttaggggaaattgacaactttgtcgcgttcatactgcactggtaactttttagagatcaagatgtgtgttttaaactggattcgtagactgttttttgaaggatgggctataaacagatgatcttcatatcttttcatagcatgtaataataattaaaaaacaattattaattactaggggaaaggagtgttcgttctacccagggtaccacagttccccacagtcaaaacccaaaagcaaggagatgagttgaaagacagtttttctttaagtcatcagtatgggatgtcagcagaacaaaaattaaaaagattaattttccttttgatctaaaacttccttagtttgagcagtaggtgctacaaaattatttacatatcttagtatcatagttaaatgtaatgtgtttaggagaggaaaacaaaagatacatttgctttaaattcattaagaaattttcaaattcactttgtagcccatgctgatagaattgggctgtgttggtacatttgaaacactgtttatgttgcttgaaacacttatttatttaatcgccgatgtgatgatgcctatggccgagatcaaatatagctagattggctagactacttatttgtttacttaaactatgggaagaagcatattattgtgtcattctgttgtgtgtgtatgtgtatatacaatataaatatatatatataaagttattttttctttgggttaatttattataagttgtaacacttggctagttttgtttgtatatgtcttaaaatgttttcttatgatatttaagtgacagttaaagaggtatcaaggtaacttgtgtagaactattctttgatatattgtcatgtttgttgtgaatattttttcttactgcacagtagaaaaataaaaacaactgagtctttattttaatgtaactcagattggggaaaacaaaacagagctaagggaacaaaatgactgagggagcactctcccacgtccagtgcactgatcattttagtatgtttgtgctttgtacggttatatatttaaaacgaaaacaaaacaaaaaaatacaagggttcatgctcttccctgggtaatagaaacagttactcgctatgcataatctagttgatagttaaatttgctattgcttttcttgtcttgttatataaaatcttttcaatacaagtttagtcttaatggtaataaaacgttatggttatttataacttgtgcttattttgtgcattttttcccatgctgaacccactaagtgcatgtagacaggactgttgttttcacactgaaaaggcaaactttgtagtagtcgttgtagtggtagacagataacgaataccaaggctgcatcatagactcctcctttaaattttttttctgtttttttttcctcttttcggttttggatataacaccagatttcagttcagagaacactcgttcaacattcagggaaagctttttacgtcacctgctatgaatgaacgtagtttgctggcaaagttttgatgcatttgctaagcattagtgggaaaggcatgccaaaatcttctctataatgtgttcaatcttgggggaaaaaaaaaggaaaaaaaatcttaggaccaggcagttgtatactttagttattaatgaatgacttcatgttaatcttgctagtttagatgatttccaagggaaagtattgtaaatgtttttttttcataatcttgttgtgtttgaattatttgtactttatctgtccagacaataaatgaaagtgtgtagaatgga

(19) United States
(12) Patent Application Publication
(10) Pub. No.: US 2026/0125679 A1
(43) Pub. Date: May 7, 2026

(54) FINE-TUNING INFLAMMATION BY ALTERING 3'UTR LENGTH

(71) Applicant: The Trustees of Columbia University in the City of New York, New York, NY (US)

(72) Inventors: Hachung CHUNG, New York, NY (US); Heegwon SHIN, New York, NY (US); Tyler DORRITY, New York, NY (US)

(21) Appl. No.: 19/400,788

(22) Filed: Nov. 25, 2025

Related U.S. Application Data
(63) Continuation of application No. PCT/US2024/031356, filed on May 29, 2024.
(60) Provisional application No. 63/469,895, filed on May 31, 2023.

(51) Int. Cl. C12N 15/113; A61K 38/17; A61K 48/00; A61P 25/28; A61P 29/00; A61P 31/14; C07K 16/18; C12N 15/86

(52) U.S. Cl. CPC C12N 15/113; A61K 38/1709; A61K 48/005; A61P 25/28; A61P 29/00; A61P 31/14; C07K 16/18; C12N 15/86; C07K 2317/76; C12N 2310/14; C12N 2310/531; C12N 2740/15043

(57) ABSTRACT

Compositions and methods for treating neurodegenerative disease or neuroinflammatory condition in a subject with HuB-effector agents.

### CROSS-REFERENCE TO RELATED APPLICATIONS

This application is a continuation of PCT International Application No. PCT/US2024/031356, filed May 29, 2024, which claims benefit of U.S. Provisional Application No. 63/469,895, filed May 31, 2023, the contents of each of which are hereby incorporated by reference.

### STATEMENT OF GOVERNMENTAL INTEREST

This invention was made with government support under NS127802 awarded by the National Institutes of Health. The government has certain rights in the invention.

### REFERENCE TO SEQUENCE LISTING

This application incorporates-by-reference nucleotide sequences which are present in the file named “93597-7402_92147-A-PCT-A_Substitute_Sequence_Listing_AWG.xml”, which is 20,002 bytes in size, and which was created on Jan. 3, 2026 in the IBM-PC machine format, having an operating system compatibility with MS-Windows, which is contained in the XML file filed Jan. 5, 2026 as part of this application.

### BACKGROUND

The disclosures of all publications, patents, patent application publications and books referred to in this application are hereby incorporated by reference in their entirety into the subject application to more fully describe the art to which the subject invention pertains.

Viruses generate lengthy double-stranded RNA (dsRNA) structures, and our innate immune system uses pattern recognition receptors (PRRs), expressed in all cell types, to detect this dsRNA and trigger antiviral defense. For instance, the cytosolic RIG-I-like receptors (RLR) (i.e., RIG-I, MDA5) and the endosomal TLR3 are PRRs that sense viral dsRNA and induce expression of the antiviral cytokine type I interferon (IFN) (1). Binding of type I IFN to its cognate interferon-α/β receptor (IFNAR) induces hundreds of interferon-stimulated gene (ISG) products with antiviral and inflammatory activities. Alternatively, some PRRs, such as PKR, detect viral dsRNA and have direct antiviral function by inducing global translational shutdown to limit virus replication (1).

Interestingly, aberrant PRR activation is also observed in non-infectious diseases, most prominently in many neurodegenerative diseases. For instance, an elevated IFN signature and PKR activation is widely observed in patient brains of amyotrophic lateral sclerosis (ALS), Alzheimer's disease, and Huntington's disease (2-7). However, the molecular events that trigger sterile inflammation in the brain are still unclear. Another stark example of sterile neuroinflammation is found in patients of Aicardi-Goutieres syndrome (AGS). AGS is a type I interferonopathy and a rare Mendelian disorder in which spontaneous type I IFN signaling occurs without acute viral infection; hence, the pathology is a mimic of viral infection (8). The various genes mutated in AGS patients share a common function: the encoded proteins are involved in RNA or DNA binding or processing. A significant proportion of these mutations cause endogenous (self) dsRNAs to be mistaken as viral dsRNAs, triggering PRR activation and downstream antiviral and inflammatory responses (8-11). What is puzzling is that genes mutated in AGS patients are all ubiquitously expressed with minimal tissue specificity, but the central nervous system (CNS) is the primary site of type I IFN production (8, 12, 13). Why the brain is especially prone to sterile inflammation is a long-standing question.

### SUMMARY

Herein are disclosed compositions and methods for treating sterile neuroinflammatory and neurodegenerative pathologies.

A method of treating a neurodegenerative disease or neuroinflammatory condition in a subject comprising administering a HuB-effector agent to the subject which decreases expression of, and/or inhibits activity of, HuB in the subject, thereby treating the neurodegenerative disease or neuroinflammatory condition.

A method of reducing immunostimulatory double-stranded RNA (dsRNA) levels and/or type I interferon (IFN) expression in a neuron, the method comprising delivering a HuB-effector agent to the neuron which decreases the expression of, and/or inhibits activity of, HuB in the neuron, thereby reducing immunostimulatory double-stranded RNA (dsRNA) levels and/or type I interferon (IFN) expression in the neuron.

A method of reducing immunostimulatory double-stranded RNA (dsRNA) levels and/or type I interferon (IFN) expression in a neuron, the method comprising delivering a HuC-effector agent to the neuron which decreases the expression of, and/or inhibits the activity of, HuC in the neuron, thereby reducing immunostimulatory double-stranded RNA (dsRNA) levels and/or type I interferon (IFN) expression in the neuron.

A modified cell that has been modified to have decreased expression or decreased activity of HuB and/or HuC.

A method of treating a neurodegenerative disease or neuroinflammatory condition in a subject comprising administering to the subject the modified cell described herein to a subject.

A method of treating or preventing a viral infection or cancer in a subject comprising administering an agent to the subject which increases the presence and/or activity of, HuB, HuC, and HuD in the subject.

A pharmaceutical composition comprising an siRNA or shRNA having complementarity to an RNA encoding human HuB and a pharmaceutically acceptable carrier.

A pharmaceutical composition comprising an siRNA or shRNA having complementarity to an RNA encoding human HuC and a pharmaceutically acceptable carrier.

A pharmaceutical composition comprising an siRNA or shRNA having complementarity to an RNA encoding human HuB and an siRNA or shRNA having complementarity to an RNA encoding human HuC, and a pharmaceutically acceptable carrier.

### BRIEF DESCRIPTION OF THE DRAWINGS

FIGS. 1A-1H. Neurons have intrinsically high levels of dsRNA. (A) Comparison of dsRNA levels across multiple human cell types. Cells stained with DAPI (blue) and J2 immunostaining (green). The J2 antibody stains for dsRNA. (B) MFI quantification of (A). (C) Levels of total RNA and dsRNA compared between hESC and day 15 neurons. Total RNA (RNASelect, green) and dsRNA (J2, red). (D) Quantification of total RNA signal in (C). (E) Quantification of dsRNA signal normalized to total RNA signal in (C). (F) Immunofluorescent images of C57BL/6 mouse cortical brain, heart, and skin tissues. Tissue sections stained with the 9D5 antibody (dsRNA, red) and neuron marker (NeuN, green). (G) Quantification of dsRNA signal in (F) co-localized with NeuN. (H) Mouse cerebral cortex stained with the 9D5 antibody (dsRNA, red) and NeuN (green). White dotted circle indicates neuron. Orange dotted circle indicates non-neuronal cells. All bar graphs are mean±S.D (n=12 cells from biological triplicates in B, D, E; n=4 images from biological triplicates in G). All scale bars represent 10 μm, except for (F), which is 50 μm. (B) One-way ANOVA with Tukey corrected multiple comparisons. (D, E, G) Student's T-test, *p<0.05, **p<0.01, ***p<0.001, ****p<0.0001.

FIGS. 2A-2E. WT neurons constitutively produce tonic type I IFN through two dsRNA-sensing PRR pathways, MAVS and TRIF. (A-B) Relative levels of type I IFN (IFNβ, pan-IFNα, IFNα21) and type III IFN (IFNλ1) (A) and ISGs (IFIT1, IFI44, ANGPTL1, STAT1) mRNA (B) in diverse human cell types. HLCs (hepatocyte-like cells), cardiomyocytes, NPCs (neural progenitor cells), neurons (day 15), and motor neurons (day 15) were all derived from WT hESCs (HUES8). (C) Type I IFN (IFNβ and IFNα subtypes) expression in 54 non-diseased tissue sites analyzed by RNA-Seq. Data derived from the Genotype-Tissue Expression (GTEx) database. TPM, transcript per million. (D) qPCR of IFNβ levels in WT neurons (day 20) following doxycycline (dox) induced knockdown of the indicated immune genes using shRNA (sh). (E) qPCR of several ISGs in WT neurons (day 20) following dox-induced knockdown of MAVS. For all qPCR, RPS11 used as housekeeping gene. All quantified data shown are mean±S.D (n=3 biological replicates). (A, B) One-way ANOVA with Tukey corrected multiple comparisons. (D, E) Two-way ANOVA with Tukey corrected multiple comparisons, *p<0.05, **p<0.01, ***p<0.001, ****p<0.0001.

FIGS. 3A-3I. ADAR1 regulates dsRNA burden in diverse cell types and tonic IFN production in neurons. (A-C) WT and ADAR1 KO NPCs (derived from HUES8 cells) were differentiated to neurons using adherent monolayer culture methods. Neurons were stained for dsRNA (J2, green), a neuron marker (MAP2, red), and DAPI (blue) at different stages of differentiation (A). Bar graphs quantifying dsRNA signal (B), and IFNβ mRNA levels (C). (D-F) WT and ADAR1 KO hESCs (HUES8) were differentiated to motor neurons via embryoid bodies in cell suspension. Neurons were stained for dsRNA (J2, green), a neuron marker (TUJ1, red), and DAPI (blue) at different stages of differentiation (D). Bar graphs quantifying dsRNA signal (E), and IFNβ mRNA levels (F). (G) qPCR of IFNβ levels in ADAR1 KO neurons (day 20) following doxycycline-induced knockdown of the indicated immune genes using shRNA (sh). (H) Immunoblot measuring PKR activation in WT and ADAR1 KO neurons over time during differentiation (ADAR1 KO neurons at day 20 were too unhealthy to obtain sufficient protein). p-PKR, phosphorylated PKR. (I) Relative levels of IFNβ mRNA in WT and ADAR1 KO HEK-293T, HeLa, hESCs, NPCs, and day 15 neurons. For all qPCR, RPS11 used as housekeeping gene. All quantified data shown are mean±S.D (n=3 biological replicates in C, F, G, H; n=12 cells from biological triplicates in B, E). Scale bars represent 10 μm. Two-way ANOVA with Tukey corrected multiple comparisons, *p<0.05, **p<0.01, ***p<0.001, ****p<0.0001.

FIGS. 4A-4G Neuron-enriched proteins HuB/C/D cooperate to induce 3′UTR elongation, dsRNA levels, and inflammation. (A-C) WT HEK-293T cells were transfected with 100 ng of plasmid expressing FLAG tagged HuB, HuC, or HuD, or a combination of HuB (33 ng), HuC (33 ng), and HuD (33 ng). EV, empty vector. 3′UTR lengthening of select genes measured via qPCR (A). The long 3′UTR transcript isoform normalized to total open reading frame (ORF) containing transcripts. (B-C) Scatterplot of the PDUI (Percentage of Distal poly-A sites Usage Index) value for genes in HEK-293T_HuB/C/D (n=3 biological replicates) vs. HEK-293T_EV (n=3 biological replicates) (B) and HEK-293T_EV vs. HEK-293T_Mock (n=3 biological replicates) (C). PDUI values derived via DaPars2 analysis. Significant change in 3′UTR length was defined as a change in PDUI greater than 1.5 fold. Genes with significantly lengthened (red, increase in PDUI) and shortened (blue, decrease in PDUI) 3′UTRs are colored (False discovery rate (FDR)≤0.05). (D-G) WT, ADAR1 KO, or ADAR1/MDA5 double knockout (DKO) HEK-293T cells were transfected as in (A). (D) Immunofluorescent images of dsRNA (J2, green) and DAPI (blue) in transfected cells. (E) Quantification of dsRNA in (D). (F) Relative IFNβ mRNA levels measured by qPCR. Housekeeping gene, RPS11. (G) Immunoblot measuring PKR activation. p-PKR, phosphorylated PKR. Scale bars represent 10 μm. All quantified data shown are mean±S.D (n=5 biological replicates in A, F; n=12 cells from biological triplicates in E). (A) One-way ANOVA with Tukey corrected multiple comparisons. (E, F) Two-way ANOVA with Tukey corrected multiple comparisons, *p<0.05, **p<0.01, ***p<0.001, ****p<0.0001.

FIGS. 5A-5E. Ectopic HuB/C/D expression in HEK-293T cells protects against Sindbis virus (SINV) infection. (A-B) Effects of HuB/C/D ectopic expression on the global transcriptome. (A) Volcano plot showing differential expression of genes between WT HEK-293T_HuB/C/D (n=3 biological replicates) vs. WT HEK-293T_EV cells (n=3 biological replicates). Empty vector, EV. Upregulated genes (red; FDR K 0.001, CPM [counts per million] fold change ≥3) and downregulated genes (blue; FDR ≤0.001, CPM fold change ≤0.333) are colored. Ectopically expressed ELAVL2, -3, -4 (HuB, -C, -D) and interferon-stimulated genes (ISGs) are marked with a black border. (B) Dot plot showing gene ontology (Biological process) analysis of upregulated genes in (A). The y-axis represents different pathways, and the x-axis represents the ratio of the differentially expressed genes. Darker red dots represent more significant enrichment. The circle size indicates the number of genes enriched in the pathway. (C-E) EV, HuB, or HuB/C/D transfected WT HEK-293T cells were infected with a Sindbis virus (SINV) dual reporter (MOI=1); BFP reports for SINV genomic mRNA and GFP for SINV subgenomic mRNA. For Pre-IFNβ set, cells were pre-treated with 0.05 nM of IFNβ for 24 hours pre-infection. For Post-IFNβ set, cells were treated with 0.05 nM of IFNβ 1 hour post-infection until harvest. (C) Flow cytometry analysis of SINV infected cells (GFP and BFP double positive cells). Representative dot plots at 6 hours and 24 hours post-infection. (D-E) Bar graph showing the frequency (%) of SINV infected cells at 6 hours (D) and 24 hours (E) post-infection. Data are shown as mean±S.D (n=3 biological replicates). One-way ANOVA with Tukey corrected multiple comparisons, *p<0.05, **p<0.01, ***p<0.001, ****p<0.0001.

FIGS. 6A-6G. HuB and HuC promote long 3′UTRs, dsRNA levels, and inflammation in neurons. (A-G) Day 20 post-differentiation neurons derived from WT and ADAR1 KO hESCs were transduced with lentivirus containing the indicated doxycycline (dox) inducible shRNA. 3′UTR shortening of selected genes measured by qPCR (A). The long 3′UTR transcript isoform normalized to total open reading frame (ORF) containing transcripts. (B) Immunofluorescent images of dsRNA (J2, green) and DAPI (blue) in transduced WT neurons. Expression of dox-inducible shRNA is coupled with RFP expression (red). (C) Quantification of dsRNA in (B). (D) Relative IFNβ mRNA levels measured by qPCR. (E-G) Same as B-D above, except in ADAR1 KO neurons. For all qPCR, RPS11 used as housekeeping gene. All quantified data shown are mean±S.D (n=3 biological replicates in A, D, G; n=12 cells from biological triplicates in C, F). Two-way ANOVA with Tukey corrected multiple comparisons, *p<0.05, **p<0.01, ***p<0.001, ****p<0.0001.

FIGS. 7A-7F. HuB and HuC protect neurons from SINV, HSV-1, and ZIKA infection. (A-E) Day 20 post-differentiation WT neurons were transduced with lentivirus containing doxycycline (dox) inducible shRNA against HuB and HuC, and then were infected with various viruses. (A-B) Neurons were infected with a Sindbis GFP reporter virus (SINV) (MOI=0.1). (A) Fluorescent and brightfield images of neurons at 24 hours post-infection. (B) Quantification of GFP-positive area in (A). (C-D) Neurons were infected with a Herpes simplex 1 (HSV1) GFP reporter virus (MOI=1). (C) Fluorescent and brightfield images of neurons at 24 hours post-infection. (D) Quantification of GFP-positive area in (C). (E) Neurons were infected with Zika virus (ZIKV) (MOI=0.1) and infection was measured via qPCR of Zika RNA at both 24 (circle) and 48 (triangle) hours post-infection. (F) Summary graphic showing the main findings of this study. For ZIKA RNA qPCR, 18S RNA used as a housekeeping gene. All quantified data shown are mean±S.D (n=3 experimental replicates). Scale bars represent 400 μm. (B, D) One-way ANOVA with Tukey corrected multiple comparisons. (E) Two-way ANOVA with Tukey corrected multiple comparisons, *p<0.05, **p<0.01, ***p<0.001, ****p<0.0001.

FIGS. 8A-8B. 3′UTR elongation in neurons and the neuron-enriched expression of ELAVL2, -3, and -4 (HuB, -C, -D). (A) Scatterplot of the PDUI (Percentage of Distal poly-A sites Usage Index) value for genes in day 16 post-differentiation neurons (n=3) vs. HEK-293T cells (n=3). Genes with significantly lengthened (red) or shortened (blue) 3′UTRs in neurons, were defined as genes with at least a 1.5 fold higher or lower average PDUI value in neurons compared to HEK-293T cells, respectively (False discovery rate (FDR)≤0.05). (B) qPCR analysis of ELAVL family member expression in HEK-293T, hESC, and day 15 post-differentiation neurons. ELAVL1 (HuR) is ubiquitously expressed, while ELAVL2, -3, and -4 (HuB, -C, -D) are enriched in neurons. All quantified data shown are mean±S.D (n=3). One-way ANOVA with Tukey corrected multiple comparisons, *p<0.05, **p<0.01, ***p<0.001, ****p<0.0001.

FIGS. 9A-9D. Ectopic expression of HuB, HuC, and HuD in HEK-293T: Total transcript levels of the 3′UTR lengthened genes remain predominantly constant. (A) Western blot demonstrating expression of FLAG tagged HuB, -C, -D in HEK-293T cells. EV, empty vector. (B) qPCR data showing total transcript expression of genes in FIG. 4A. Primers detect a region in the open reading frame (ORF). RPS11 used as a housekeeping gene for qPCR analysis. (C) Volcano plot showing differential expression of genes between HEK-293T_EV (n=3) vs. HEK-293T_HuB/C/D cells (n=3). Genes whose 3′UTR is lengthened (dark red, light red) or shortened (dark blue, light blue) in HEK-293T_HuB/C/D cells are colored, which was determined by DaPars analysis in FIG. 4B; dark color dots indicate genes that are also significantly differentially expressed (CPM [counts per million] fold change ≥3, FDR ≤0.001). Note that most genes with altered 3′UTR length do not exhibit significant differences in transcript expression levels. Genes with no significant change in 3′UTR length and expression levels are in gray. (D) Distribution of sequencing reads for select genes whose 3′UTR is lengthened in day 16 (d16) post-differentiation neuron and HEK-293T_HuB/C/D cells. Numbers on y-axis indicate RNA seq read coverage. All quantified data shown are mean±S.D (n=3). One-way ANOVA with Tukey corrected multiple comparisons, *p<0.05, **p<0.01, ***p<0.001, ****p<0.0001.

FIGS. 10A-10D: Knockdown of ELAVL proteins in neurons reduces PKR activation and ISG expression (A) Western blot demonstrating effective knockdown of HuB, HuC, and HuD in neurons using doxycycline (dox) inducible shRNAs. (B-C) Western blot measuring PKR activation in WT (B) and ADAR1 KO (C) neurons with indicated ELAVL protein depleted. p-PKR, phosphorylated PKR. (D) qPCR of ISGs (IFIT1 and IFI44) in WT and ADAR1 KO neurons following depletion of the indicated ELAVL proteins. All quantified data shown are mean±S.D (n=3). Two-way ANOVA with Tukey corrected multiple comparisons, *p<0.05, **p<0.01, ***p<0.001, ****p<0.0001.way ANOVA with Tukey corrected multiple comparisons, *p<0.05, **p<0.01, ***p<0.001, ****p<0.0001.

FIG. 11: Depletion of HuB can prolong the survival of ADAR1 KO neurons (A) Brightfield images of ADAR1 KO neurons with doxycycline-inducible knockdown of HuB, -C, or -D at 15, 20, 25, and 30 days post differentiation. Images are representative of three biological triplicates. Scale bars represent 400 μm.

### DETAILED DESCRIPTION

A method of treating a neurodegenerative disease or neuroinflammatory condition in a subject comprising administering a HuB-effector agent to the subject which decreases expression of, and/or inhibits activity of, HuB (for example, SEQ ID NO:1) in the subject, thereby treating the neurodegenerative disease or neuroinflammatory condition.

In embodiments, the HuB-effector agent is delivered to a brain or central nervous system of the subject.

In embodiments, the HuB-effector agent is delivered to neurons, preferably differentiated neurons, preferably post-mitotic neurons, of the subject.

In embodiments, the decrease in the expression of, or inhibition of the activity of, HuB occurs in neurons of the subject, preferably differentiated neurons, preferably post-mitotic neurons.

In embodiments, the HuB-effector agent comprises a small interfering RNA (siRNA), a short hairpin RNA (shRNA), a CRISPR RNA (crRNA), a single-guide RNA (sgRNA), and/or a prime editing guide RNA (pegRNA) which has complementarity to a nucleotide molecule encoding HuB, preferably a gene or transcript which comprises at least one portion encoding HuB.

In embodiments, the HuB-effector agent comprises an antibody, or portion thereof, which binds HuB, preferably wherein the antibody inhibits the activity of HuB, preferably RNA-binding activity of HuB.

In embodiments, the HuB-effector agent is delivered to the subject via adeno-associated virus (AAV)-mediated delivery, lentiviral delivery, a virus-like particle (VLP), or Sindbis viral delivery.

A method of treating a neurodegenerative disease or neuroinflammatory condition in a subject comprising administering a HuC-effector agent to the subject which decreases expression of, and/or inhibits activity of, HuC (for example, SEQ ID NO:2) in the subject, thereby treating the neurodegenerative disease or neuroinflammatory condition.

In embodiments, the HuC-effector agent is delivered to a brain or central nervous system of the subject.

In embodiments, the HuC-effector agent is delivered to neurons, preferably differentiated neurons, preferably post-mitotic neurons, of the subject.

In embodiments, the decrease in the expression of, or inhibition of the activity of, HuC occurs in neurons of the subject, preferably differentiated neurons, preferably post-mitotic neurons.

In embodiments, the HuC-effector agent comprises a small interfering RNA (siRNA), a short hairpin RNA (shRNA), a CRISPR RNA (crRNA), a single-guide RNA (sgRNA), and/or a prime editing guide RNA (pegRNA) which has complementarity to a nucleotide molecule encoding HuC, preferably a gene or transcript which comprises at least one portion encoding HuC.

In embodiments, the HuC-effector agent comprises an antibody, or portion thereof, which binds HuC, preferably wherein the antibody inhibits activity of HuC, preferably RNA-binding activity of HuC.

In embodiments, the HuC-effector agent is delivered to the subject via adeno-associated virus (AAV)-mediated delivery, lentiviral delivery, a virus-like particle (VLP), or Sindbis viral delivery.

In embodiments, the methods further comprise administering to said subject a HuC-effector agent according to the methods herein.

In embodiments, the neurodegenerative or neuroinflammatory disease is Aicardi-Goutieres syndrome (AGS), amyotrophic lateral sclerosis (ALS), Parkinson's disease, or Alzheimer's disease. In embodiments, the neurodegenerative or neuroinflammatory disease is Aicardi-Goutieres syndrome (AGS). In embodiments, the neurodegenerative or neuroinflammatory disease is amyotrophic lateral sclerosis (ALS). In embodiments, the neurodegenerative or neuroinflammatory disease is Parkinson's disease or Alzheimer's disease.

In embodiments, the subject is further administered a preventative or prophylactic antiviral treatment.

A method of reducing immunostimulatory double-stranded RNA (dsRNA) levels and/or type I interferon (IFN) expression in a neuron, the method comprising delivering a HuB-effector agent to the neuron which decreases the expression of, and/or inhibits activity of, HuB (for example, SEQ ID NO:1) in the neuron, thereby reducing immunostimulatory double-stranded RNA (dsRNA) levels and/or type I interferon (IFN) expression in the neuron.

In embodiments, an average 3′UTR length of transcripts expressed by the neuron is decreased, preferably wherein the neuron displays a decreased average Percentage of Distal poly-A sites Usage Index (PDUI) value relative to a non-treated neuron.

In embodiments, MDA5-mediated type I interferon production by the neuron is decreased.

In embodiments, Protein Kinase R (PKR) activation in the neuron is decreased.

In embodiments, the neuron is in culture or in a brain of a subject.

In embodiments, the neuron is derived from an isolated cell, preferably a stem cell, a neural stem cell (NSC), an induced pluripotent stem cell (iPSC), or a human embryonic stem cell (hESC).

In embodiments, the isolated cell is isolated from a subject suffering from or at risk of developing a neurodegenerative disease or neuroinflammatory condition, preferably wherein the neurodegenerative disease or neuroinflammatory condition is Aicardi-Goutieres syndrome (AGS), amyotrophic lateral sclerosis (ALS), Parkinson's disease, or Alzheimer's disease.

In embodiments, the decrease in the expression of, or inhibition of the activity of, HuB occurs in differentiated neurons or post-mitotic neurons, preferably after the neuron has terminally differentiated.

In embodiments, the HuB-effector agent comprises a small interfering RNA (siRNA), a short hairpin RNA (shRNA), a CRISPR RNA (crRNA), a single-guide RNA (sgRNA), and/or a prime editing guide RNA (pegRNA) which has complementarity to a nucleotide molecule encoding HuB, preferably a gene or transcript which comprises at least one portion encoding HuB.

In embodiments, the HuB-effector agent comprises an antibody, or portion thereof, which binds HuB, preferably wherein the antibody inhibits the activity of HuB, preferably RNA-binding activity of HuB.

In embodiments, the HuB-effector agent is delivered to the subject via adeno-associated virus (AAV)-mediated delivery, lentiviral delivery, a virus-like particle (VLP), or Sindbis viral delivery.

In embodiments, type I interferon (IFN) expression comprises IFN-beta and/or IFN-alpha expression.

A method of reducing immunostimulatory double-stranded RNA (dsRNA) levels and/or type I interferon (IFN) expression in a neuron, the method comprising delivering a HuC-effector agent to the neuron which decreases the expression of, and/or inhibits the activity of, HuC (for example, SEQ ID NO:2) in the neuron, thereby reducing immunostimulatory double-stranded RNA (dsRNA) levels and/or type I interferon (IFN) expression in the neuron.

In embodiments, the average 3′UTR length of transcripts expressed by the neuron is decreased, preferably wherein the neuron displays a decreased average Percentage of Distal poly-A sites Usage Index (PDUI) value relative to a non-treated neuron.

In embodiments, MDA5-mediated type I interferon production by the neuron is decreased.

In embodiments, Protein Kinase R (PKR) activation in the neuron is decreased.

In embodiments, the neuron is in culture or in a brain of a subject.

In embodiments, the neuron is derived from an isolated cell, preferably a stem cell, a neural stem cell (NSC), an induced pluripotent stem cell (iPSC), or a human embryonic stem cell (hESC).

In embodiments, the isolated cell is isolated from a subject suffering from or at risk of developing a neurodegenerative disease or neuroinflammatory condition, preferably wherein the neurodegenerative disease or neuroinflammatory condition is Aicardi-Goutieres syndrome (AGS), amyotrophic lateral sclerosis (ALS), Parkinson's disease, or Alzheimer's disease.

In embodiments, the decrease in the expression of, or inhibition of the activity of, HuC occurs in differentiated neurons or post-mitotic neurons, preferably after the neuron has terminally differentiated.

In embodiments, the HuC-effector agent comprises a small interfering RNA (siRNA), a short hairpin RNA (shRNA), a CRISPR RNA (crRNA), a single-guide RNA (sgRNA), and/or a prime editing guide RNA (pegRNA) which has complementarity to a nucleotide molecule encoding HuC, preferably a gene or transcript which comprises at least one portion encoding HuC.

In embodiments, the HuC-effector agent comprises an antibody, or portion thereof, which binds HuC, preferably wherein the antibody inhibits the activity of HuC, preferably the RNA-binding activity of HuC.

In embodiments, the HuC-effector agent is delivered to the subject via adeno-associated virus (AAV)-mediated delivery, lentiviral delivery, a virus-like particle (VLP), or Sindbis viral delivery.

In embodiments, type I interferon (IFN) expression comprises IFN-beta and/or IFN-alpha expression.

In embodiments, the methods further comprise delivering to said neuron a HuC-effector agent according to the methos herein.

A modified cell that has been modified to have decreased expression or decreased activity of HuB (for example, SEQ ID NO:1) and/or HuC (for example, SEQ ID NO:2).

In embodiments, the cell is a neuron. In embodiments, the cell is a mammalian CNS neuron.

In embodiments, the neuron is derived from an isolated cell, preferably a stem cell, a neural stem cell (NSC), an induced pluripotent stem cell (iPSC), or a human embryonic stem cell (hESC).

In embodiments, the decrease in the expression of, or inhibition of the activity of, HuB and/or HuC occurs in the neuron after the neuron has differentiated, preferably after the neuron has terminally differentiated, or is a post-mitotic neuron.

A method of treating a neurodegenerative disease or neuroinflammatory condition in a subject comprising administering to the subject the modified cell described herein to a subject.

In embodiments, the cell is administered to the patient by autologous engraftment or allogenic engraftment.

A method of treating or preventing a viral infection or cancer in a subject comprising administering an agent to the subject which increases the presence and/or activity of, HuB, HuC, and HuD (for example, SEQ ID NO:3) in the subject.

In embodiments, the methods comprise administering to the subject HuB, HuC, and/or HuD proteins, or variants thereof, or administering to the subject one or more nucleic acid molecules that express HuB, HuC, and/or HuD, or variants thereof.

In embodiments, the HuB, HuC, and/or HuD proteins and/or one or more nucleic acid molecules are delivered to the subject via adeno-associated virus (AAV)-mediated delivery, lentiviral delivery, a virus-like particle (VLP), or Sindbis viral delivery.

In embodiments, the subject is susceptible to infection with a herpes simplex virus, Zika virus, Sindbis virus, and/or a flavirius.

A pharmaceutical composition comprising an siRNA or shRNA having complementarity to an RNA encoding human HuB and a pharmaceutically acceptable carrier.

A pharmaceutical composition comprising an siRNA or shRNA having complementarity to an RNA encoding human HuC and a pharmaceutically acceptable carrier.

A pharmaceutical composition comprising an siRNA or shRNA having complementarity to an RNA encoding human HuB and an siRNA or shRNA having complementarity to an RNA human HuC, and a pharmaceutically acceptable carrier.

A method of treating a sterile inflammatory neurodegenerative condition in a subject comprising administering an agent to the subject which downregulates expression of, or activity of, HuB so as to treat the sterile inflammatory neurodegenerative condition.

A method of reducing immunostimulatory dsRNA levels in a brain of a subject comprising administering an agent to the subject which downregulates expression of, or activity of, HuB so as to reduce immunogenic dsRNA levels in the brain of the subject.

In embodiments, the methods further comprise administering a second agent which downregulates expression of or activity of HuC.

A method of treating a sterile inflammatory neurodegenerative condition in a subject comprising administering an agent to the subject which downregulates expression of, or activity of, HuC so as to treat the sterile inflammatory neurodegenerative condition.

In embodiments, the sterile inflammatory neurodegenerative condition comprises Aicardi-Goutieres syndrome or amyotrophic lateral sclerosis.

In embodiments, the sterile inflammatory neurodegenerative condition comprises Alzheimer's disease or Parkinson's disease.

In embodiments, the agent comprises an siRNA or shRNA having complementarity to an RNA encoding human HuB.

In embodiments, the agent, or second agent, comprises an siRNA or shRNA having complementarity to an RNA encoding human HuC

In embodiments, the agent is delivered to the central nervous system of the subject via adeno-associated virus (AAV)-mediated delivery.

In embodiments, the agent inhibits activity of human HuB.

In embodiments, the agent inhibits activity of human HuC.

In embodiments, the agent is an antibody which binds to and reduces the RNA-binding activity of human HuB.

In embodiments, the agent is an antibody which binds to and reduces the RNA-binding activity of human HuC.

In embodiments, the subject is a human subject 21 years or older.

A pharmaceutical composition comprising an siRNA or shRNA having complementarity to an RNA encoding human HuB, an siRNA or shRNA having complementarity to an RNA encoding human HuC, and a pharmaceutically acceptable carrier.

A pharmaceutical composition comprising an siRNA or shRNA having complementarity to an RNA encoding human HuB and a pharmaceutically acceptable carrier.

A pharmaceutical composition comprising an siRNA or shRNA having complementarity to an RNA encoding human HuC and a pharmaceutically acceptable carrier.

Exemplary embodiments of a HuB protein sequence are provided by NCBI GenPept Accession No. NP_004423 and SEQ ID NO: 1, as well as related isoforms and variants thereof. Exemplary embodiments of nucleic acid sequences which encode a HuB protein include an ELAVL2 gene (e.g., NCBI Gene ID: 1993) or transcript (e.g., NCBI GenBank Accession No. NM_004432, SEQ ID NO: 4), as well as related isoforms or variants thereof.

Exemplary embodiments of a HuC protein sequence are provided by NCBI GenPept Accession No. NP_001411 and SEQ ID NO: 2, as well as related isoforms and variants thereof. Exemplary embodiments of nucleic acid sequences which encode a HuC protein include an ELAVL3 gene (e.g., NCBI Gene ID: 1995) or transcript (e.g., NCBI GenBank Accession No. NM_001420, SEQ ID NO: 5), as well as related isoforms or variants thereof.

Exemplary embodiments of a HuD protein sequence are provided by NCBI GenPept Accession No. NP_001138246 and SEQ ID NO: 3, as well as related isoforms and variants thereof. Exemplary embodiments of nucleic acid sequences which encode a HuD protein include an ELAVL4 gene (e.g., NCBI Gene ID: 1996) or transcript (e.g., NCBI GenBank Accession No. NM_001144774, SEQ ID NO: 6), as well as related isoforms or variants thereof.

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

Definitions: The terms used in this specification generally have their ordinary meanings in the art, within the context of this invention and the specific context where each term is used. Certain terms are discussed herein to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means a mammal. Mammals include canines, felines, rodents, bovine, equines, porcines, ovines, and primates including humans. Thus, in embodiments the compositions and/or methods can be used in human medicine or also in veterinary medicine, e.g., to treat companion animals, farm animals, laboratory animals in zoological parks, and animals in the wild. In embodiments the compositions and/or methods are particularly desirable for human medical applications. In a preferred embodiment the subject is a human.

The terms “treat”, “treatment” of a disease or condition, and the like refer to slowing down, relieving, ameliorating or alleviating at least one of the symptoms of the condition.

The terms “therapeutically effective amount” or “amount effective to” encompasses, unless otherwise indicated, an amount sufficient to ameliorate or inhibit a symptom or sign of the medical condition. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Herein is disclosed that mammalian neurons carry exceptionally high levels of dsRNA that stimulates PRR mediated inflammation in homeostasis and disease. Additionally, we demonstrate that the neuron-enriched ELAVL family of genes (ELAVL2, -3, and -4) lengthen 3′UTRs, and elongated 3′UTRs serve as a major source for immunostimulatory dsRNA structures in neurons. Neuronal dsRNAs can serve as a major upstream trigger for inflammation in the brain.

Loss of RNA homeostasis underlies numerous neurodegenerative and neuroinflammatory diseases. However, the molecular mechanisms that trigger neuroinflammation are poorly understood. Viral double-stranded RNA (dsRNA) triggers innate immune responses when sensed by host pattern recognition receptors (PRRs) present in all cell types. Here, we report that human neurons intrinsically carry exceptionally high levels of immunostimulatory dsRNAs and identify long 3′UTRs as giving rise to neuronal dsRNA structures. We found that the neuron-enriched ELAVL family of genes (ELAVL2, -3, -4) can increase 1) 3′UTR length, 2) dsRNA load, and 3) activation of dsRNA sensing PRRs (e.g., MDA5, PKR, TLR3). In wild-type neurons, neuronal dsRNAs signaled through PRRs to induce tonic production of the antiviral type I interferon. Depleting ELAVL2 in WT neurons led to global shortening of 3′UTR length, reduced immunostimulatory dsRNA levels, and rendered WT neurons susceptible to herpes simplex virus and Zika virus infection. Neurons deficient in ADAR1, a dsRNA-editing enzyme mutated in the neuroinflammatory disorder Aicardi-Goutieres syndrome (AGS), exhibited intolerably high levels of dsRNA that triggered PRR mediated toxic inflammation and neuronal death. Depleting ELAVL2 in ADAR1 knockout neurons led to prolonged neuron survival by reducing immunostimulatory dsRNA levels. In summary, neurons are specialized cells where PRRs constantly sense ‘self’ dsRNAs to pre-emptively induce protective antiviral immunity, but maintaining RNA homeostasis is paramount to prevent pathological neuroinflammation.

Neurons have Intrinsically High Levels of dsRNA

We used the J2 antibody to image dsRNAs in various human cell types (14). The J2 antibody, which binds long dsRNAs ≥40 bp independent of sequence, has been used for detecting both viral and endogenous dsRNAs (14-17). We focused on human cells since endogenous dsRNA levels and identity is distinct from species to species, even between mouse vs. human (18, 19). We differentiated wildtype (WT) HUES8 human embryonic stem cells (hESCs) to neural progenitor cells (NPCs) and neurons using well-established adherent monolayer culture methods (see Materials and Methods) (20, 21). After confirming the identity of cell types using various lineage markers, we conducted J2 immunostaining. Interestingly, J2 antibody staining levels dynamically changed at different stages of hESC differentiation, with neurons having the noticeably strongest J2 signal. In parallel, we also derived hepatocyte-like cells (HLCs) from the same parental hESC line (22). HLC identity was confirmed by staining for various lineage markers. HLCs had a much dimmer J2 signal than hESCs. Similarly, neurons derived from an independent hESC line (WA09) also had a much higher dsRNA burden than hESCs and HLCs.

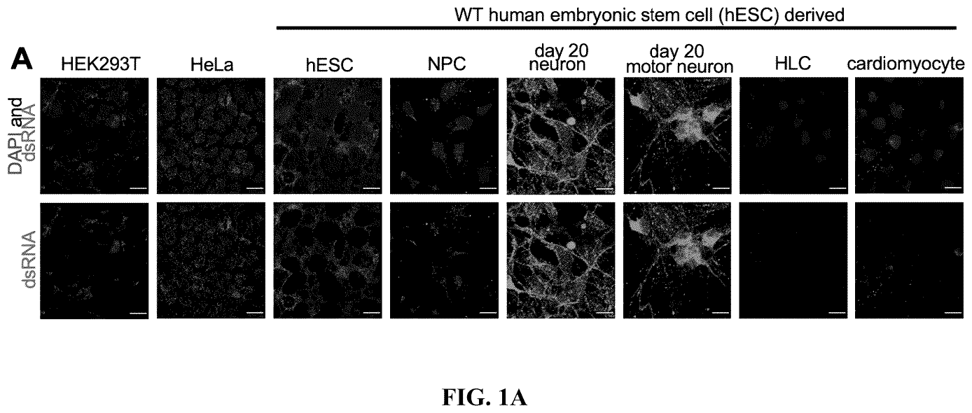

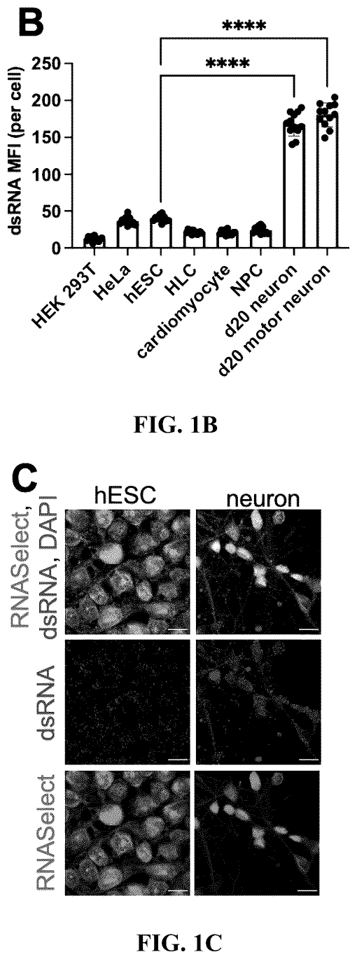

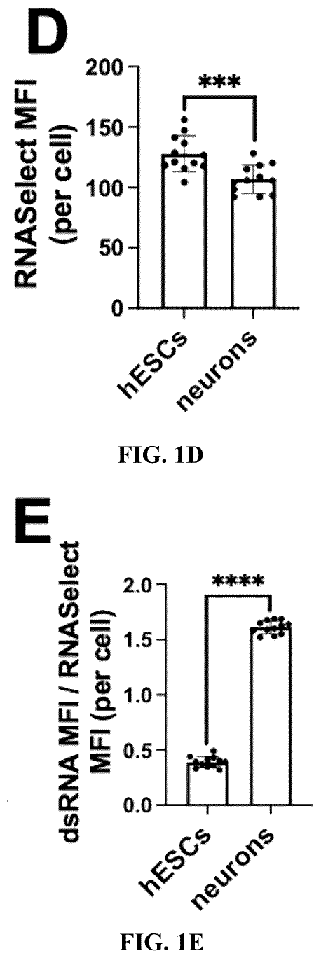

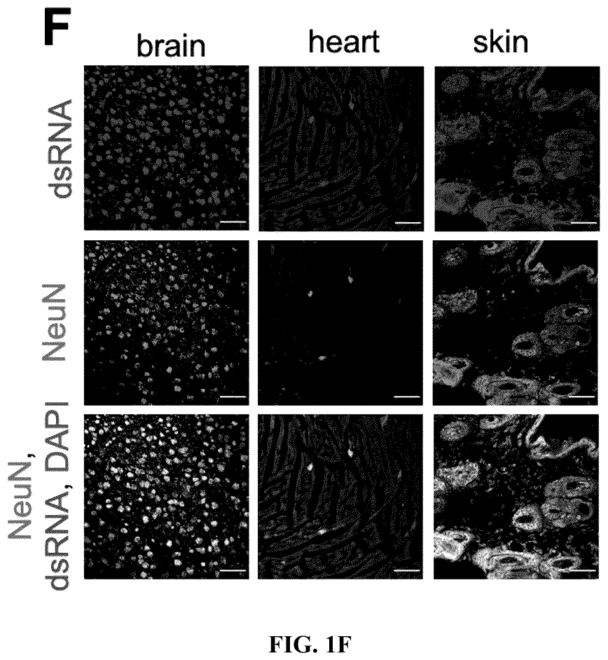

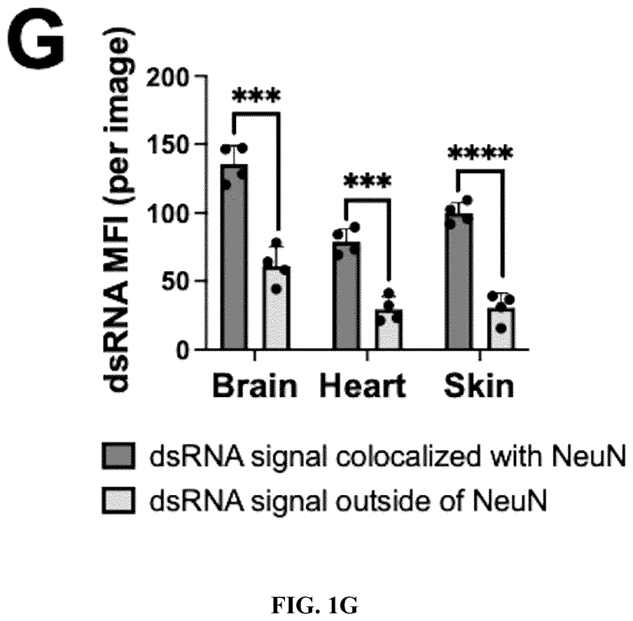

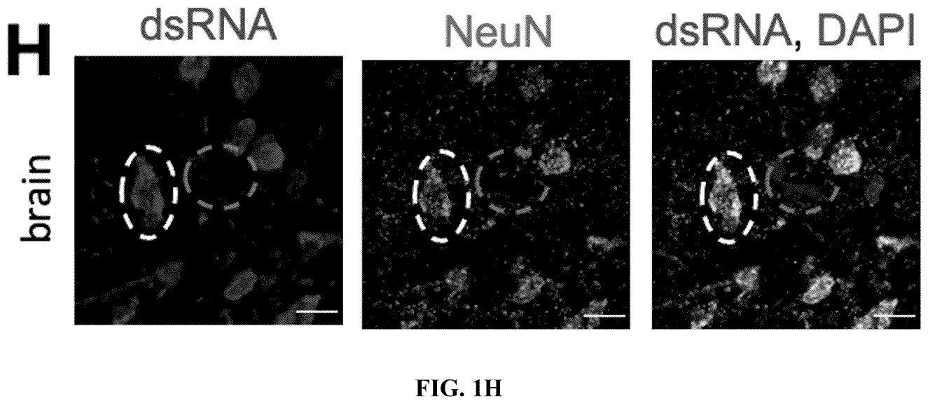

We next compared dsRNA levels side-by-side among a broader range of cell types. We generated specialized motor neurons, which are derived via embryoid bodies in cell suspension as opposed to adherent monocultures (23). We also generated cardiomyocytes, a post-mitotic cell type, to compare to post-mitotic neurons and analyzed HEK-293T and HeLa cell lines as non-hESC derived controls. Neurons had strikingly higher dsRNA levels than any other tested cell type (FIG. 1A, 1). Additionally, dsRNA subcellular localization also significantly differed by cell type. Neuronal dsRNA signal was present throughout the nucleus and cytoplasm, while in hESCs, dsRNA was mainly in the cytoplasm.

We further confirmed J2 specificity for dsRNA and validated the above observations using different antibodies. We demonstrated that the J2 signal was sensitive to treatment with a double-stranded RNase (dsRNAse), but not a single-stranded RNase (ssRNAse) or a and confirmed that other anti-dsRNA antibodies such as 9D5 and K1 yielded results similar to J2. Additionally, by staining cells with an anti-GAPDH antibody, we confirmed that cell type differences in J2 antibody staining are not due to mere differences in cell permeability to antibody. While neurons still exhibited a significantly stronger dsRNA stain than other cell types, neurons did not show a higher GAPDH stain.

We next examined whether neurons had more dsRNA simply because they had more total RNA than other cell types. In parallel to J2, we co-stained total RNA using RNASelect. RNASelect staining showed that total RNA load is, in fact, slightly lower in neurons than in hESCs (FIG. 1C, 1D). Importantly, we normalized the J2 signal (dsRNA load) to the RNASelect signal (total RNA load) from the same images and observed that normalized J2 signal is much higher in neurons than in hESCs (FIG. 1E). We also performed polyA FISH (fluorescence in situ hybridization) (24), which selectively stains for polyadenylated mRNA, and observed that neurons have slightly less mRNA than hESCs. Thus, the increase in dsRNA levels in neurons is not due to an increase in total RNA levels. Instead, the neuronal transcriptome appears to be prone to forming dsRNA structures.

Next, we examined if neurons are enriched for dsRNAs in complex tissue. We co-stained tissue from 10-month old C57BL/6 mice for dsRNA and NeuN, a pan-neuronal marker. Since the J2 antibody is a mouse monoclonal antibody, we stained dsRNA in mouse tissue with the rabbit monoclonal 9D5 antibody, and confirmed that 9D5 staining in mouse tissue is sensitive to dsRNAse treatment, but not ssRNAse or DNAse treatment. We stained brain tissue and other innervated organs such as the heart and skin. Remarkably, we found that dsRNA signal is much stronger in cells co-stained with NeuN than in cells without NeuN (FIG. 1F, 1G). Interestingly, NeuN⁺ cells in the periphery, such as in the skin and heart, also contain high levels of dsRNA similar to NeuN⁺ cells in the brain; although further experimentation is necessary to determine if peripheral NeuN⁺ cells are tissue-innervating nerves or another cell type. Additionally, in the mouse cerebral cortex, NeuN⁺ cells, indicative of neurons, had a higher dsRNA burden than nearby non-neuronal cells (NeuN⁻) (FIG. 1H). Thus, neurons are one of the most enriched cell types for dsRNA in complex tissue, and this phenotype is conserved across mouse and human cells.

Neurons are Enriched for dsRNA Originating from POLII

Prior studies demonstrated that mRNA non-coding regions (e.g., introns, 3′UTRs) or mitochondrial RNAs (mtRNAs) can contribute to cellular dsRNAs (15-19). We transiently treated neurons with different RNA polymerase inhibitors for 12 hours prior to J2 staining to determine if RNA polymerase II (POLII) or mitochondrial RNA polymerase (POLRMT) give rise to neuronal dsRNA. The following inhibitors were used: actinomycin D (ActD), which inhibits both POLII and POLRMT (16); α-amanitin, a POLII inhibitor; and IMT1, a POLRMT-specific inhibitor (25). We confirmed the specificity of the inhibitors in neurons and hESCs: ActD down-regulated both mtRNA and mRNA levels, while α-amanitin and IMT1 down-regulated levels of mRNA and mtRNA, respectively. For both cell types, 12 hours of ActD treatment was sufficient to abrogate almost all dsRNA signal, revealing that cellular dsRNAs are present transiently—no longer than 12 hours. Additionally, a dual treatment with α-amanitin and IMT1 also completely reduced the dsRNA signal, suggesting that the majority of dsRNA consists of POLII- and POLRMT-derived transcripts. By quantifying depletion of dsRNA upon single treatments with α-amanitin or IMT1 treatment, we calculated the proportional contribution of POLII- or POLRMT-derived dsRNAs, respectively. We applied this analysis to various cell types. In HeLa cells, roughly 90% of dsRNA was derived from POLRMT. This is consistent with a prior study that concluded that roughly 95% of dsRNA in HeLa cells is located in mitochondria (16), validating our analysis. In both hESCs and HEK-293T cells, POLRMT and POLII contribute to roughly 70% and 30% of dsRNA, respectively. Conversely, in neurons, POLRMT and POLII transcripts contribute to 40% and 60% of dsRNA, respectively. We further validated these results by imaging colocalization of the dsRNA with the mitochondria and observed less percentage of dsRNA colocalized in the mitochondria in neurons compared to hESCs. In summary, mtRNA is the major component of dsRNA in HeLa, HEK-293T, and hESCs. However, in neurons, POLII-derived transcripts, presumably mRNAs, contribute to the bulk of the neuronal dsRNA load.

WT Neurons Constitutively Produce IFN Through dsRNA-Sensing Pathways

Past studies noted that type I IFN can be constitutively expressed at low quantities, yet could have profound functions in homeostasis (26). Using SIMOA, an ultra-sensitive ELISA assay, a recent study reported the intriguing observation that human neurons constitutively produce IFNβ (27). Additionally, studies using IFNAR-deficient model systems proposed a model in which constitutive type I IFN in the brain may be required to pre-empt viral infection, prevent neurodegeneration, and promote synaptic plasticity (27-29). These ideas align with the concept that inflammation exists on a spectrum—rather than being just active during infection or injury—and is involved in a broad range of biological processes beyond its classical role in antimicrobial defense (30).

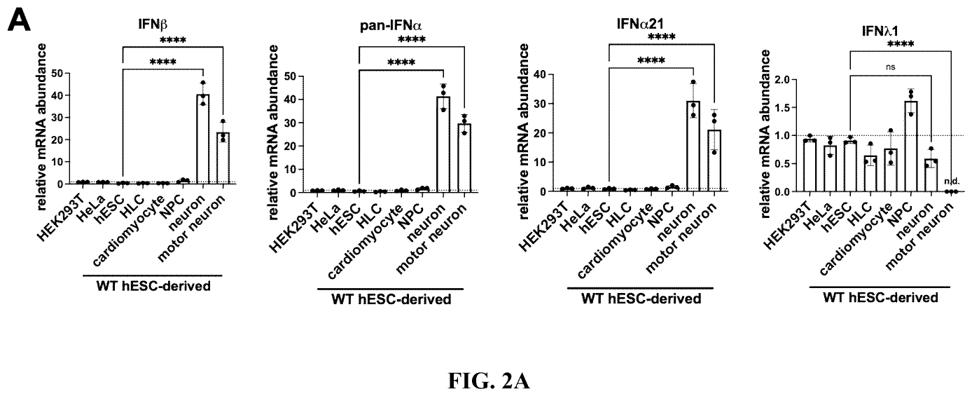

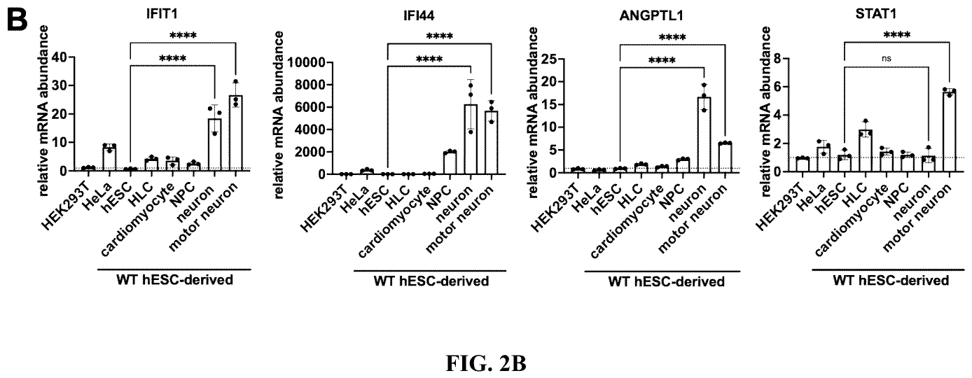

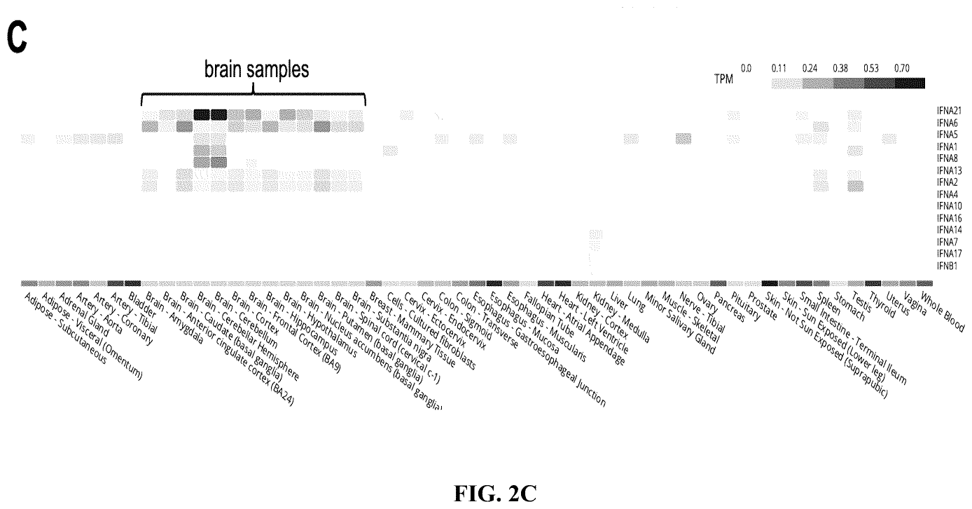

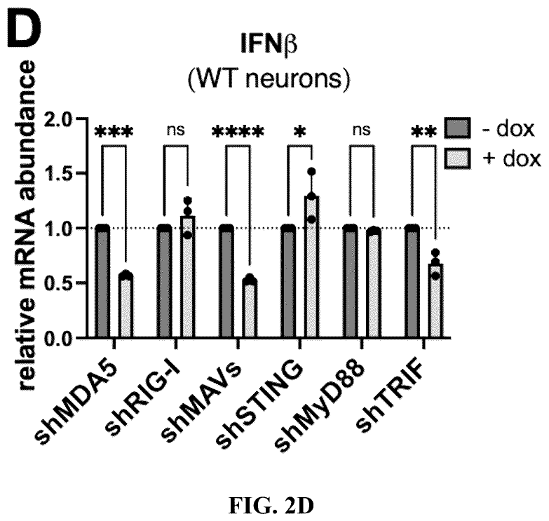

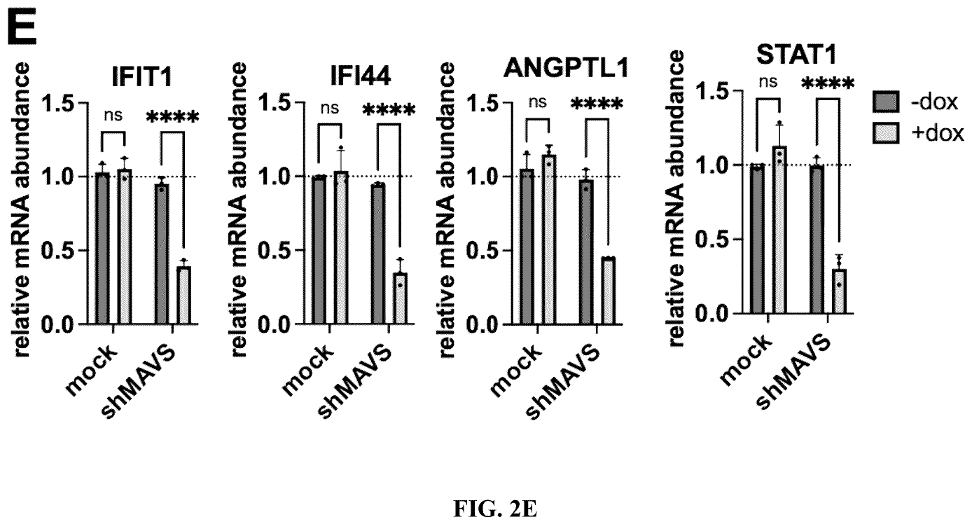

Supporting the possibility that the brain constitutively expresses ‘tonic’ type I IFN, we observed via qPCR that neurons derived from WT hESCs produced much higher levels of basal type I IFN (IFNβ and IFNα subtypes) mRNA compared to a panel of other human cell types (FIG. 2A). Type I IFN mRNA was minimally or not detected in the other human cell types. We did not observe an up-regulation of IFNλ1, a type III IFN (FIG. 2A). We also examined the levels of several ISGs to determine if the tonic type I IFN in neurons was sufficient to induce ISG expression. Overall, ISGs such as IFIT1, IFI44, and ANGPTL1 were expressed highest in neurons (FIG. 2B). However, some ISGs, like STAT1, were not expressed the highest in all neurons (FIG. 2B). Since tonic type I IFN is expressed at low levels—drastically lower than viral induced type I IFN—and since many ISGs are expressed at baseline in a tissue-specific manner independently of IFN, we do not expect that neurons have the highest expression of all ISGs. Taken together, these data suggest that a low-grade tonic type I IFN is expressed in hESC-derived neurons.

It is unknown if the human brain also exhibits a constitutive type I IFN signature. We analyzed the Genotype-Tissue Expression (GTEx) Portal (31) derived from nearly 1000 individuals, and found that the non-diseased human brain expressed much higher levels of type I IFN compared to many other tissues (FIG. 2C). Interestingly, many IFNα subtypes were uniquely elevated in the human brain, most notably IFNα21, which was also strongly induced in stem cell-derived neurons (FIG. 2A). In addition to the GTEx dataset, we also analyzed RNA-Seq datasets from the Human Protein Atlas (HPA) derived from 198 individuals (32). Analysis of the HPA RNA-Seq dataset shows that three compartments display a notable type I IFN signature: the human brain, bone marrow and lymphoid tissue, and the male epididymis. Hence, the brain is one of the compartments in the human body that constitutively expresses type I IFN in the absence of acute infection. Of note, when we analyzed publicly available single cell RNA-Seq datasets, we could not detect significant type I IFN expression in the non-diseased human brain; likely because low abundance transcripts such as tonic type I IFN mRNA is not effectively captured by single cell RNA-Seq.

To investigate the mechanisms underlying constitutive type I IFN expression, we tested if neuronal dsRNAs constantly activate PRRs to maintain tonic IFN in homeostasis. This hypothesis diverges from the classical view that PRRs distinguish self from non-self ligands. We transduced neurons with lentiviruses carrying doxycycline inducible shRNAs that target four immune adaptor proteins (STING, MAVS, MyD88, and TRIF) that lie downstream of PRRs and license transcription factors to induce transcription of type I IFN genes (33). STING is downstream of DNA-sensing cGAS, MAVS is downstream of the dsRNA-sensing RLRs MDA5 and RIG-I, MyD88 is downstream of various TLRs that sense a variety of ligands, and TRIF is downstream of the dsRNA-sensing TLR3. We confirmed successful knockdown of each adaptor protein in neurons. Notably, we found that constitutive IFNβ production in WT neurons is reduced following knockdown of either MAVS or TRIF—the two adaptor proteins implicated in dsRNA sensing—but not STING or MyD88 (FIG. 2D). We also confirmed that reducing IFNb levels by knocking down MAVS, led to reduced ISG induction, suggesting that dsRNA sensing pathways sustain basal ISG expression in neuronal homeostasis (FIG. 2E). As tonic IFNβ production does not rely on MyD88, this suggests that ssRNA sensors like TLR7/8 are dispensable for tonic IFNβ production. The reduction in constitutive IFNβ following TRIF knockdown is consistent with a prior investigation, where cortical neurons derived from induced pluripotent stem cells of patients with defective TLR3 had reduced constitutive type I IFN expression (27). When comparing the two RLRs, MDA5 knockdown, but not RIG-I knockdown, significantly reduced IFNβ levels (FIG. 2D).

We also asked if there was a positive correlation between expression levels of dsRNA-sensing PRRs and constitutive IFN production. MDA5, RIG-I, and TLR3 mRNA expression was much lower in neurons compared to a panel of other human cell types, suggesting that it is not high levels of PRRs that confer the ability to constitutively express IFN in neurons. Instead, high dsRNA load in neurons drives tonic type I IFN production and ISG expression via the cytoplasmic MDA5-MAVS and endosomal TLR3-TRIF signaling pathways.

Loss of ADAR1 increases dsRNA levels in diverse cell types, and neurons fail to tolerate loss of ADAR1

Next, we examined the consequences of enriched dsRNA in neurons in a disease model. AGS is an encephalopathy and genetic disorder where the brain is the primary site for inflammation (8, 12, 13). ADAR1 is a dsRNA editing enzyme that converts Adenosine (A)-to-Inosine (I) in dsRNA, and is one of the genes mutated in AGS patients (34, 35). Past investigations demonstrated that ADAR1 deficiency in mammals leads to aberrant activation of dsRNA sensing PRRs such as MDA5 and PKR (9, 10, 36-42). This led to the prevailing model that ADAR1 editing of self-dsRNAs introduces mismatches that disrupts dsRNA structures, thereby suppressing self-dsRNAs from triggering dysregulated PRR activation. However, this model is largely based on RNA structure prediction and in vitro biochemical studies and has yet to be conclusively demonstrated. Moreover, like other genes mutated in AGS, ADAR1 is ubiquitously expressed with low tissue specificity; thus, it was puzzling why the brain is the primary site for type I IFN production in AGS.

We reasoned that the high levels of dsRNA in neurons predispose the brain to pathological inflammation in ADAR1 deficiency. Hence, we tested if loss of ADAR1 leads to an increase in total dsRNA levels and whether ADAR1 deficiency would induce toxic inflammation in neurons, but not other cell types with lower intrinsic dsRNA burden. ADAR1 defective mouse models exhibit multiorgan systemic inflammation, some of which also display AGS-like encephalopathy (43, 44). However, overall it has been challenging for AGS mouse models to phenocopy neurologic disease and CNS-centric inflammation (45-47). Additionally, because mice lack Alu elements—repetitive elements prone to forming dsRNA structures that are heavily edited by human ADAR1—only ˜0.004% of edited sites in human are conserved in mice (48, 49). Hence, we used ADAR1 knockout (KO) hESCs (9) to obtain NPCs and neurons. Previous attempts to obtain neurons from ADAR1 KO hESCs failed, due to cell death at the NPC stage (9). However, by optimizing differentiation conditions to achieve extremely synchronized cell populations, we were able to derive ADAR1 KO NPC and neurons successfully (see Materials and Methods).

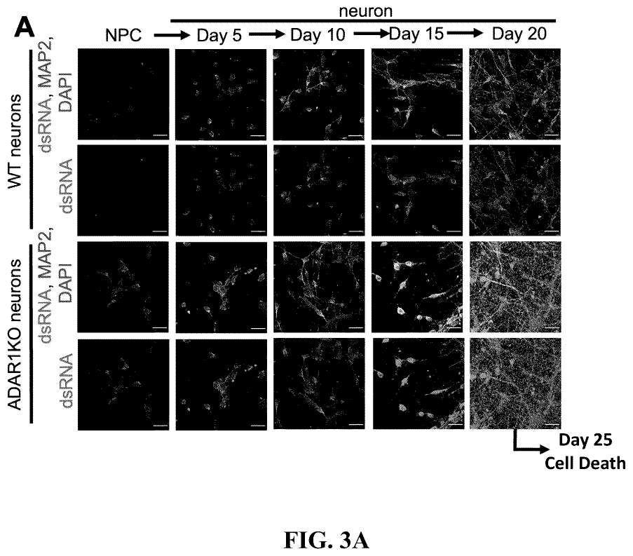

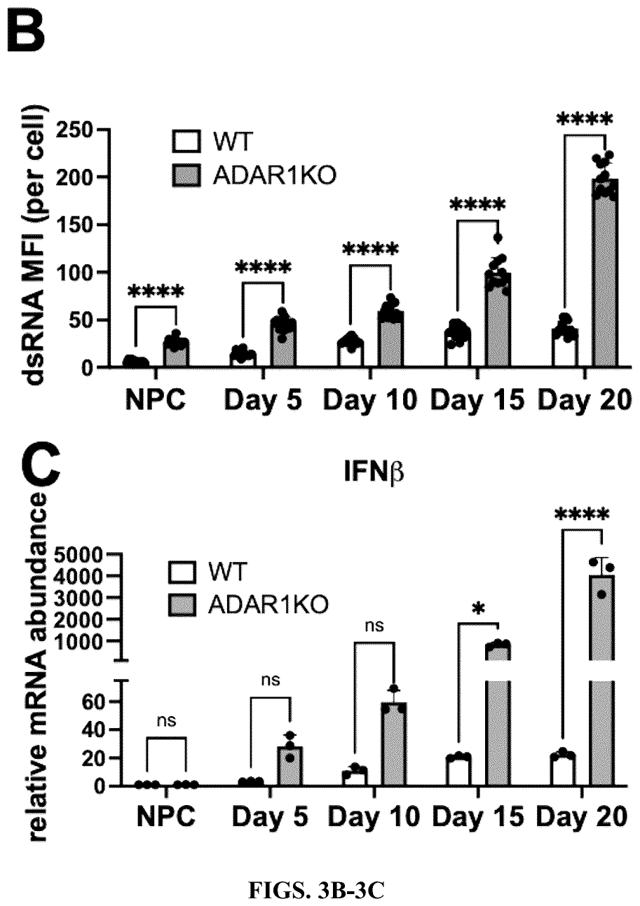

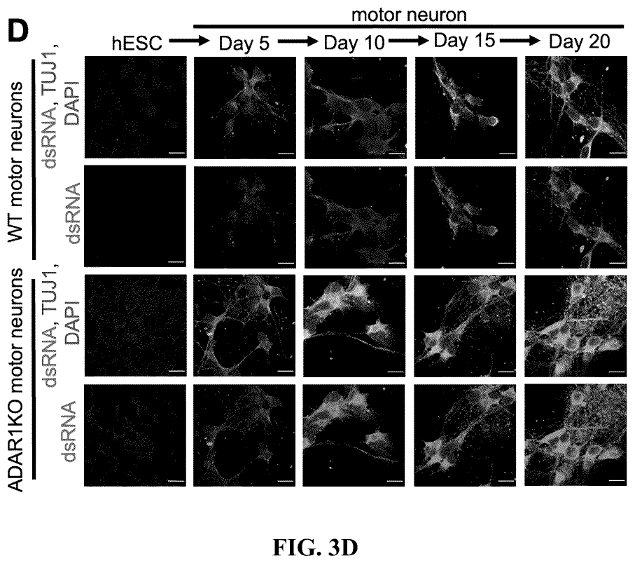

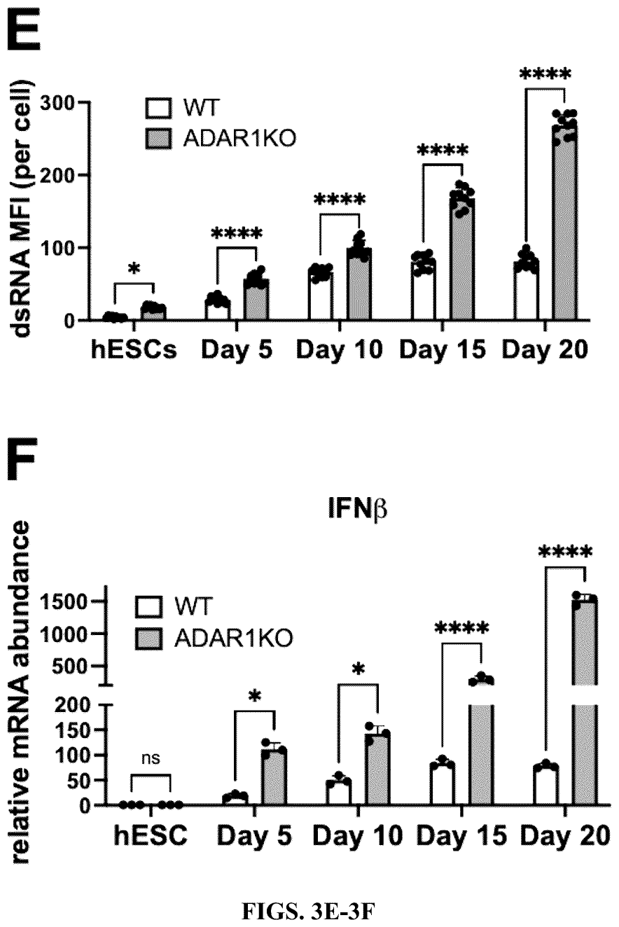

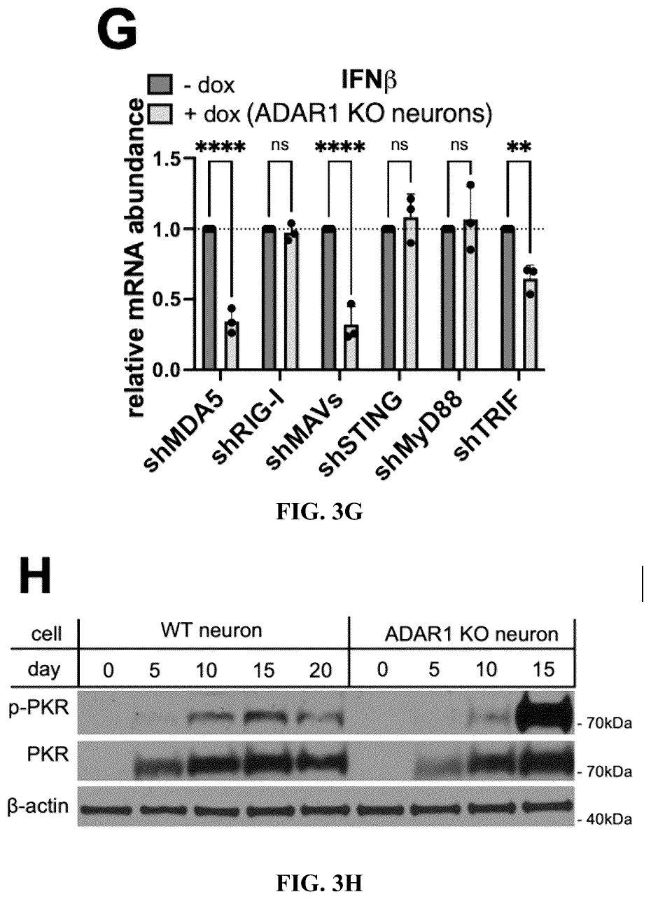

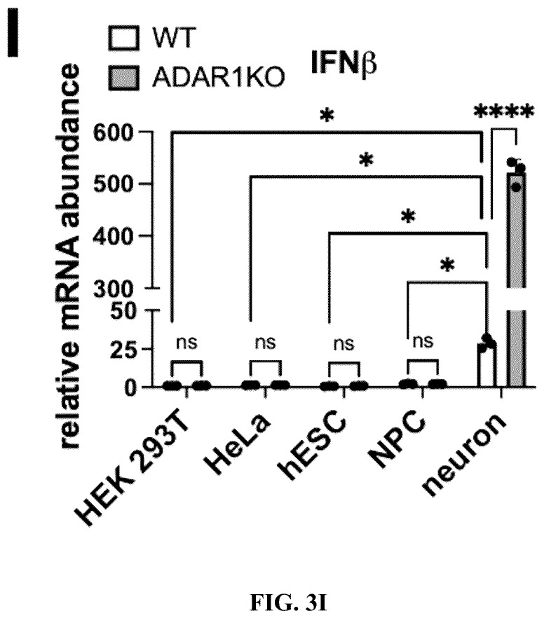

We confirmed that loss of ADAR1 did not interfere with NPC or neuron differentiation (FIG. 3A). Importantly, dsRNA and type I IFN production in ADAR1 KO neurons increased in an uncontrolled manner, unlike WT neurons where dsRNA and type I IFN levels are increased, but plateau at 15˜20 days post-differentiation (FIG. 3A, 3B, 3C). By day 20 post-differentiation, ADAR1 KO neurons had extremely high levels of dsRNA (FIG. 3A, 3B)—resembling an RNA virus infection—and IFNβ production in ADAR1 KO neurons increased up to more than 100-fold compared to IFNβ levels in WT neurons (FIG. 3C). This increase in IFNβ production corresponded with an increase in the production of ISGs. We also differentiated WT and ADAR1 KO hESCs to specialized motor neurons to further validate the above findings (23). ADAR1 KO hESCs had no defect in differentiating into motor neurons (FIG. 3D). dsRNA and IFNβ levels were highly dysregulated in ADAR1 KO motor neurons, but not in WT motor neurons (FIG. 3D, 3E, 3F).

Similar to tonic type I IFN in WT neurons, dysregulated type I IFN production in ADAR1 KO neurons also depended on MDA-MAVS and TLR3-TRIF pathways (FIG. 3G). Therefore, the pathways to produce low/tonic and high/dysregulated type I IFN production are shared. Additionally, this report demonstrates that ADAR1 can suppress signaling via TRIF, an unexpected finding since ADAR1 is nuclear and cytoplasmic, and TLR3-TRIF sensing occurs in endosomes.

We observed that ADAR1 KO neurons die by day 25 post-differentiation, and therefore sought to understand the mechanism of cell death. We knocked down MAVS to decrease IFNβ production, which resulted in a modest increase in cell survival, indicating that dsRNA-induced IFNβ only modestly contributes to cell death in ADAR1 KO neurons. Additionally, we knocked down PKR to observe its role in cell death. PKR is an ISG and a PRR that undergoes autophosphorylation upon binding dsRNA and inhibits translation. In addition to aberrant IFN production, ADAR1 KO neurons also exhibit hyperactivation of PKR (FIG. 3H). Knockdown of PKR did not alter IFNβ production. Yet, PKR depletion was able to significantly increase survival of ADAR1 KO neurons. Together, these data indicate that PKR activation and IFNβ production play a major and minor role in ADAR1 KO neuron death, respectively.

Finally, it is important to note that lack of ADAR1 led to elevated dsRNA levels in all cell types examined (hESCs, NPCs, neurons) (FIG. 3B, 3E), but only in neurons did ADAR1 deficiency lead to markedly increased type I IFN levels (FIG. 3I, 3C, 3F). Of note, previous studies into ADAR1 KO HEK-293T or HeLa cells also demonstrated that loss of ADAR1 does not induce IFN production at baseline; however inflammation can be induced in these cells following an exogenous IFN stimulus (10, 39). In summary, these data demonstrate that ADAR1 is a global regulator of dsRNA across multiple cell types, and neurons are especially susceptible to inflammation upon loss of ADAR1.

In addition to ADAR1, another catalytically active member of the ADAR family is ADAR2. Like ADAR1, ADAR2 binds to dsRNA and performs A-to-I editing, however ADAR2 is primarily known as a site-specific editor of coding regions (42). We examined dsRNA levels in ADAR2 KO HEK-293T cells (9). Although ADAR2 protein is expressed in HEK-293T cells, ADAR2 did not significantly alter dsRNA burden. Hence, ADAR1, rather than ADAR2, is the major regulator of cellular dsRNA levels.

Ectopically expressed ELAVL2, -3, -4 (HuB, -C, -D) cooperate to lengthen 3′UTRs, increase dsRNA levels, and induce inflammation

The identity of the dsRNAs that play a causal role in inflammation remains elusive. Here, we tested the hypothesis that mRNAs with elongated 3′UTRs are the main contributors to immunostimulatory dsRNAs. More than half of human genes generate alternative mRNA isoforms that differ in their 3′UTRs but encode the same protein (50, 51). Elongated 3′UTRs are thought to be versatile platforms to recruit various RNA binding proteins that can regulate transcript stability and subcellular location (51, 52). The 3′UTR is a heavily structured region in mRNA (53) and harbors many repetitive elements that can base-pair and form dsRNAs (18). The brain expresses the longest 3′UTRs of any human tissue, whereas the liver carries much shorter 3′UTRs (54, 55). Moreover, among the different CNS cell types, neurons carry the longest median 3′UTR length, even longer than glia cells (54). As expected, we observed global 3′UTR lengthening in stem cell-derived neuronal cultures. 3′UTR length was determined using DaPars2 (56), a program that uses RNA sequencing data to detect alternative polyadenylation sites and produce a PDUI (Percentage of Distal poly-A sites Usage Index) value for each gene (Data file S1). An increase in PDUI indicates 3′UTR lengthening, and a decrease in PDUI indicates 3′UTR shortening.

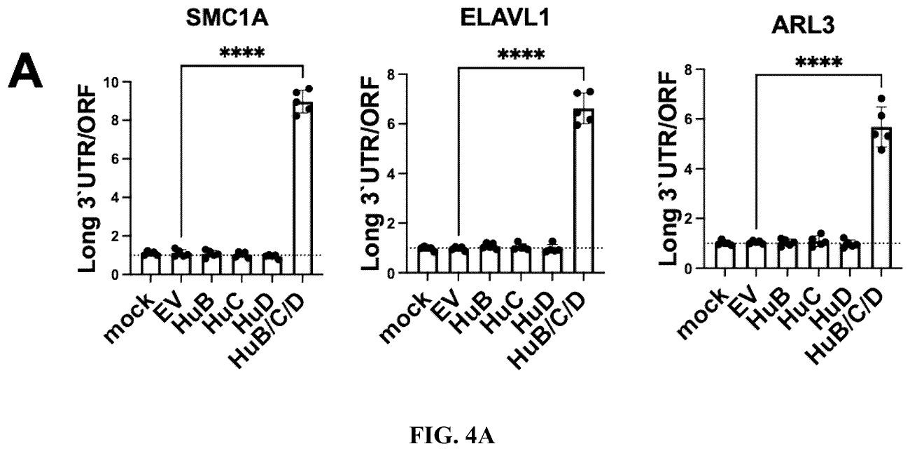

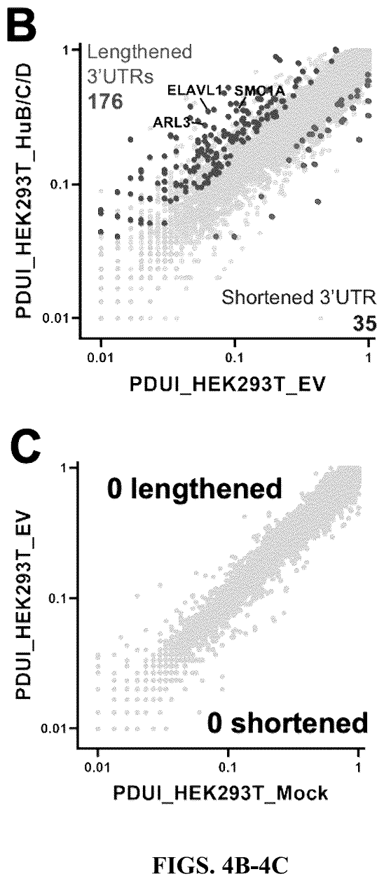

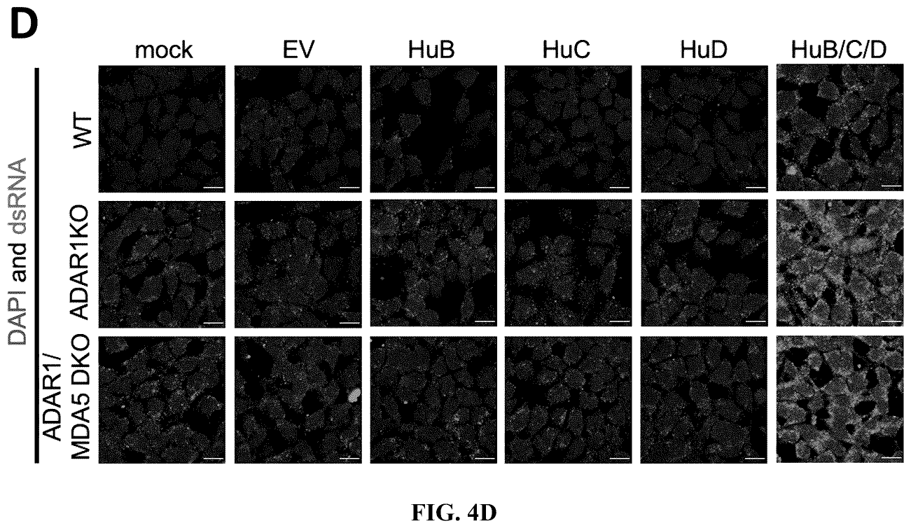

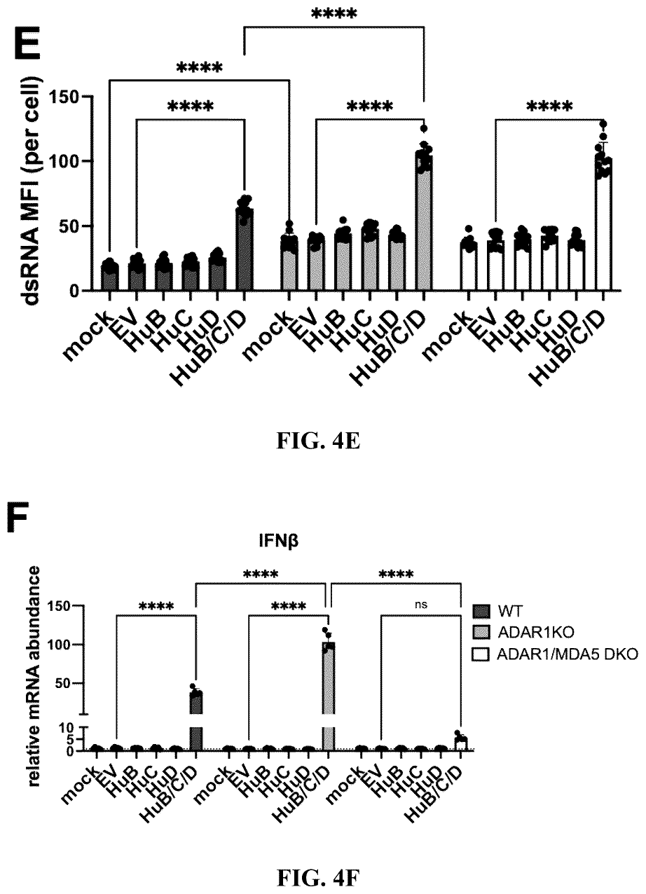

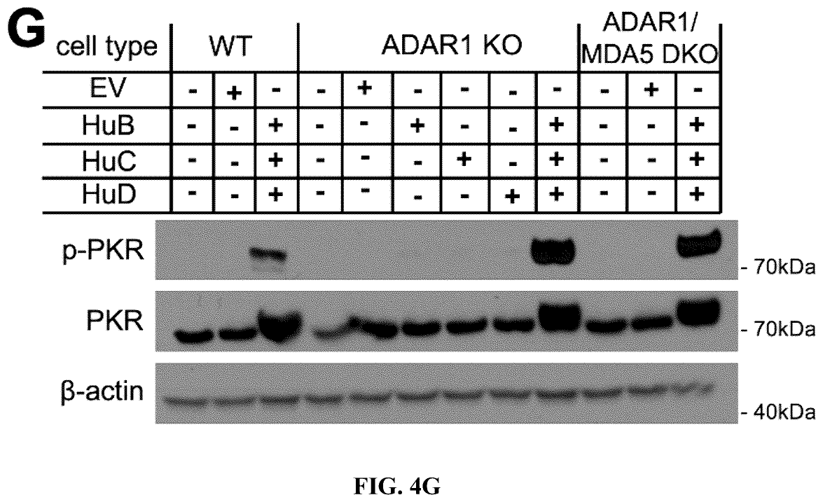

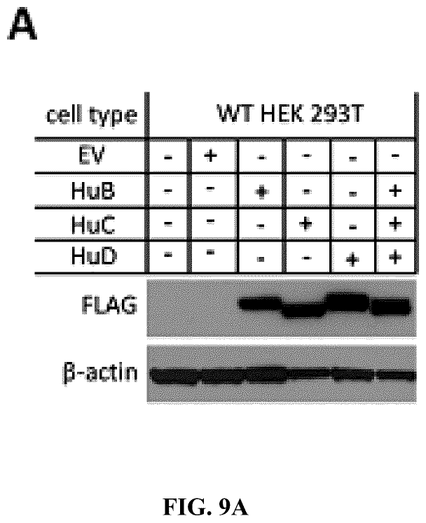

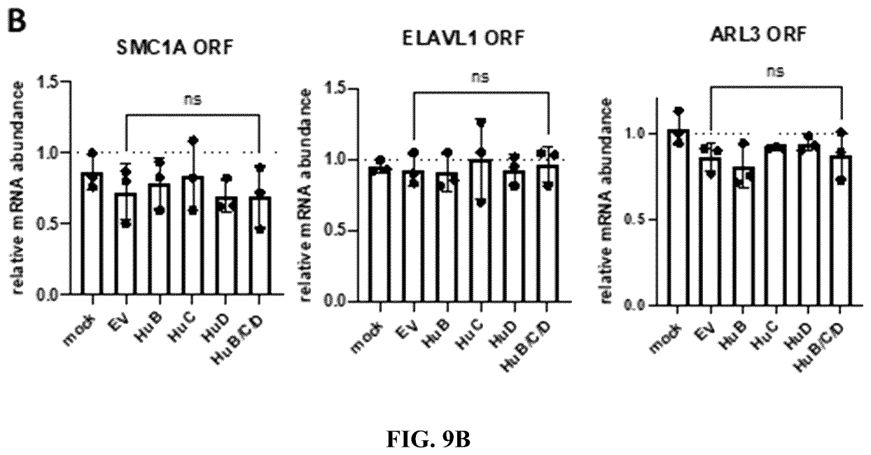

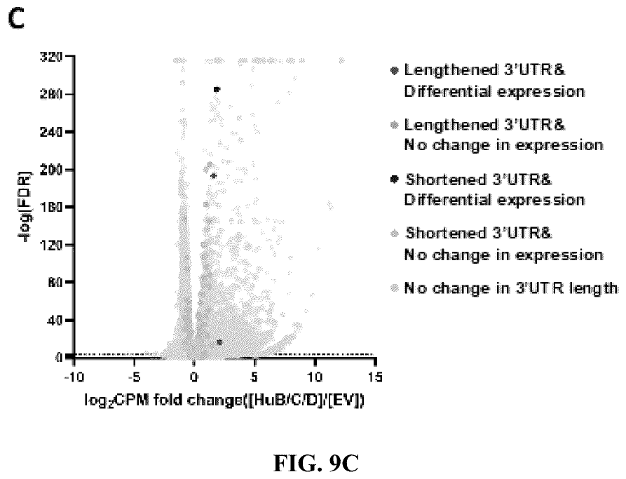

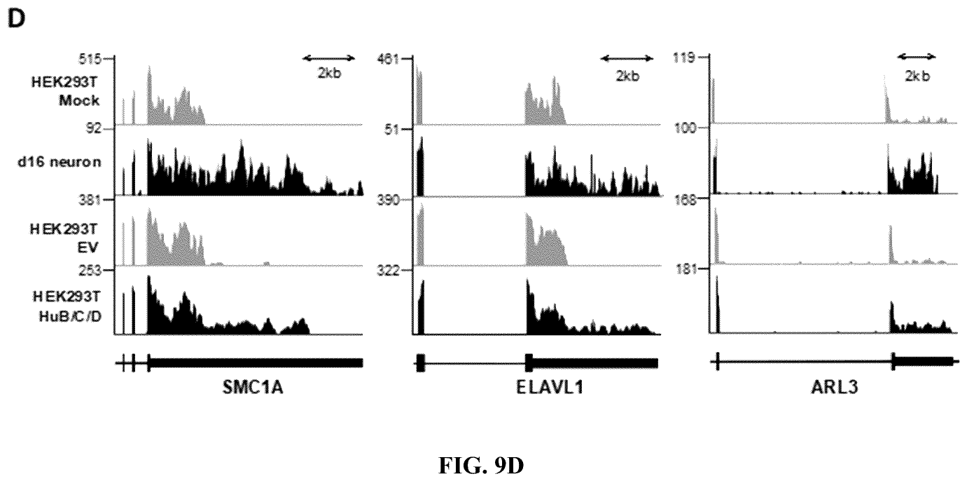

To test if elongated 3′UTRs contribute to immunostimulatory dsRNA burden, we needed a method to force elongation of 3′UTRs; however, the neural-specific mechanism of 3′UTR lengthening is not fully understood in mammals. A recent study in Drosophila identified three genes in the ELAV/Hu family (Elav, Fne, and Rbp9) that play a role in lengthening 3′UTRs in neurons (57). The human homologs of these genes are HuB (encoded by ELAVL2), HuC (encoded by ELAVL3), and HuD (encoded by ELAVL4). HuB, -C, and D are neuron-enriched RNA binding proteins that recognize AU-rich elements in the 3′UTRs and are thought to play a role in neuronal development (58-61). As expected, ELAVL2, -3, -4 transcript expression was detected in stem cell-derived neurons but minimally detected in hESCs and HEK-293T cells. We ectopically expressed FLAG-tagged HuB, HuC, or HuD in HEK-293T cells and assayed for 3′UTR lengthening of select genes. However, individual expression of HuB, HuC, or HuD failed to affect 3′UTR length (FIG. 4A). Hence, next we tested if combined expression of HuB, HuC, and HuD could synergistically lengthen 3′UTRs. We found that combined expression of HuB/C/D markedly induced 3′UTR lengthening of select genes (FIG. 4A) and globally when DaPars2 analysis was performed (FIG. 4B, 4C). Importantly, total transcript levels of the 3′UTR lengthened genes remain predominantly constant (FIG. 9). Therefore, we demonstrated that expression of HuB/C/D can induce short to long 3′UTR isoform switching in a non-neural cell type such as HEK-293T cells. Since, HuB, -C, or -D deficient mice exhibit a spectrum of phenotypes ranging from various neural defects and neonatal death (59, 61, 62), this HuB/C/D ectopic expression system allowed us to decouple the neural developmental role of HuB, -C, and -D from their other potential roles in immunity.

With our newly developed HuB/C/D ectopic expression system, we examined how 3′UTR lengthening impacted dsRNA levels and the innate immune response. At baseline, HEK-293T cells are low in dsRNA load (FIG. 1A) and express minimal IFNβ (FIG. 3I). However, triple expression of HuB/C/D led to a significant increase in dsRNA levels in both WT and ADAR1 KO HEK-293T cells compared to individual expression of each gene (FIG. 4D, 4E). In all conditions, we observed higher J2 signal in ADAR1 KO HEK-293T cells than in comparable WT cells (FIG. 4D, 4E), consistent with our prior conclusion that ADAR1 is a global regulator of cellular dsRNA load across cell types. Triple HuB/C/D expression also led to spontaneous IFNβ production in WT HEK-293T cells, and even higher levels of IFNβ production in ADAR1 KO cells (FIG. 4F). These effects mirror those in neurons: constitutive type I IFN production that is exacerbated by ADAR1 deficiency (FIG. 3C, 3F). Additionally, lack of MDA5 did not alter total dsRNA levels as expected (FIG. 4D, 4E); but did abrogate IFNβ production (FIG. 4F), suggesting that HuB/C/D-induced dsRNAs signal through MDA5 to produce type I IFN. We also checked PKR activation as another metric of spontaneous inflammation. We found that triple expression of HuB/C/D induced PKR activation in WT HEK-293T cells and ADAR1 deficiency further increased PKR activation (FIG. 4G). Taken together, expression of the three neuron enriched genes HuB/C/D can cooperatively increase 3′UTR length, dsRNA burden, and cellular inflammation mediated by MDA5 and PKR. Therefore, we conclude that elongated 3′UTRs are one of the major contributors to immunostimulatory dsRNA structures.

### Ectopic Expression of ELAVL2, -3, -4 (HuB, -C, -D) Confers Antiviral Activity.

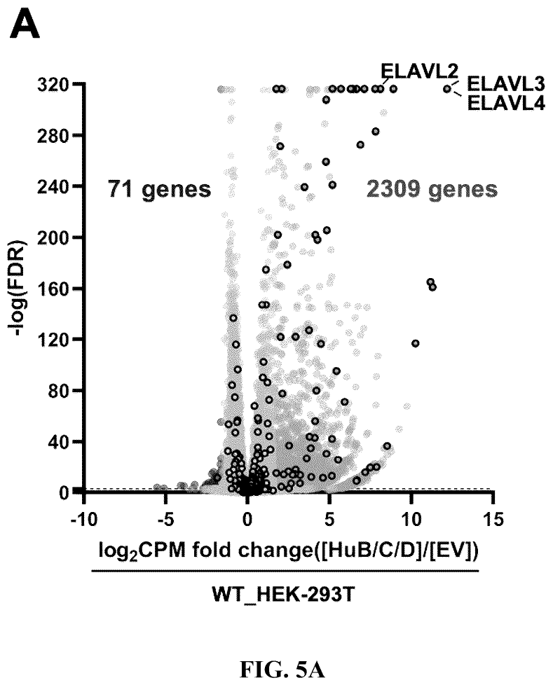

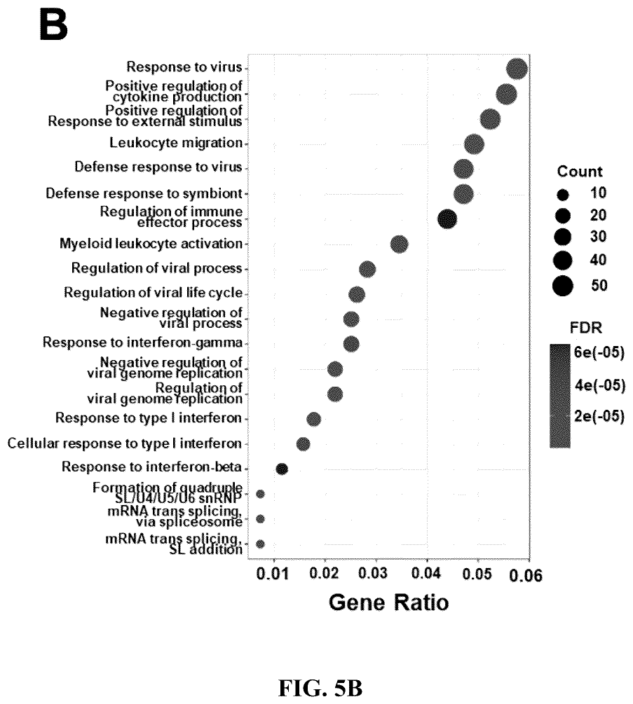

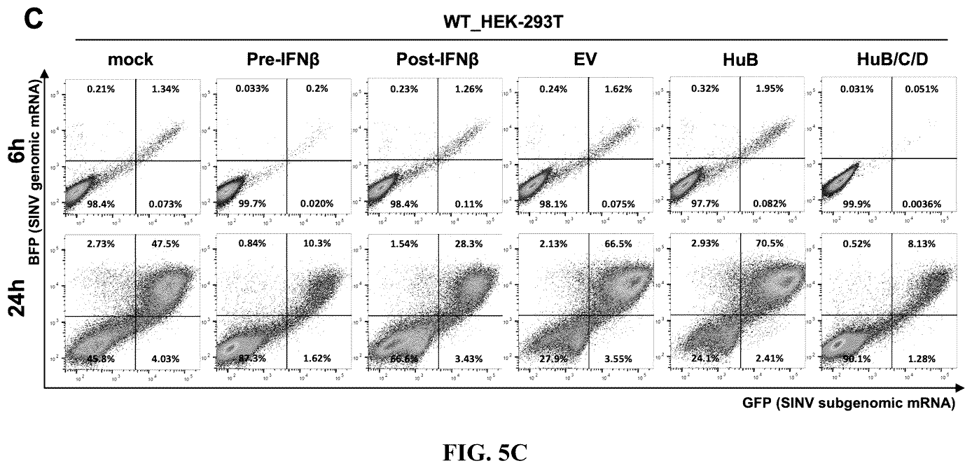

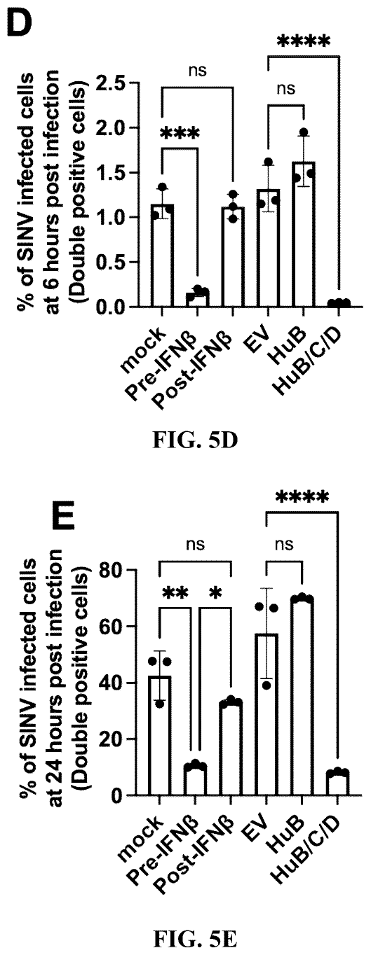

To gain a more comprehensive understanding of the consequences of HuB, -C, -D expression, we conducted a transcriptome-wide analysis of genes differentially expressed upon HuB/C/D expression in WT HEK-293T cells. Upon HuB/C/D expression, we found 2309 significantly up-regulated and 71 significantly down-regulated genes (FIG. 5A), while only 6 genes are upregulated when comparing WT HEK-293T cells expressing empty vector to the mock condition. Gene ontology analysis showed that up-regulated genes were predominantly enriched for antiviral defense and immune response pathways (FIG. 5B), and 64 of the up-regulated genes were ISGs (FIG. 5A); an indication that HuB/C/D expression induces an extensive antiviral and inflammatory signature.

Since HuB/C/D expression in WT HEK-293T cells induced type I IFN and PKR activation (FIG. 4), we postulated that HuB/C/D expression emulates a pre-emptive antiviral state that protects against viral infection. To test this idea, we expressed either empty vector, HuB alone, or HuB/C/D in WT HEK-293T cells, and then infected the cells with Sindbis virus (SINV) containing a dual reporter: BFP reports for SINV genomic mRNA, and GFP for the SINV subgenomic mRNA. Triple expression of HuB/C/D significantly reduced the percent of infected cells when compared to the empty vector or HuB alone at 6 and 24 hours post-infection (FIG. 5C, 5D, 5E). In parallel, we pre-treated cells with IFNβ 24 hours prior to infection, and found that HuB/C/D expression protects as effectively as IFNβ pre-treatment (FIG. 5C, 5D, 5E). Finally, we also treated cells with IFNβ 1 hour post-infection. While IFNβ post-treatment had some protective effect, it was not as protective as IFNβ pre-treatment or HuB/C/D expression (FIG. 5C, 5D, 5E); indicating that pre-existing IFNβ prior to viral infection has more potent antiviral activity than IFNβ induced after infection. In summary, we uncovered that HuB, -C, -D proteins—classically thought to play a neurodevelopmental role—can provide cellular resistance to viral infection.

Loss of ELAVL2 and -3 (HuB and -C) in neurons results in lower immunostimulatory dsRNA levels and enhanced susceptibility to virus infection.

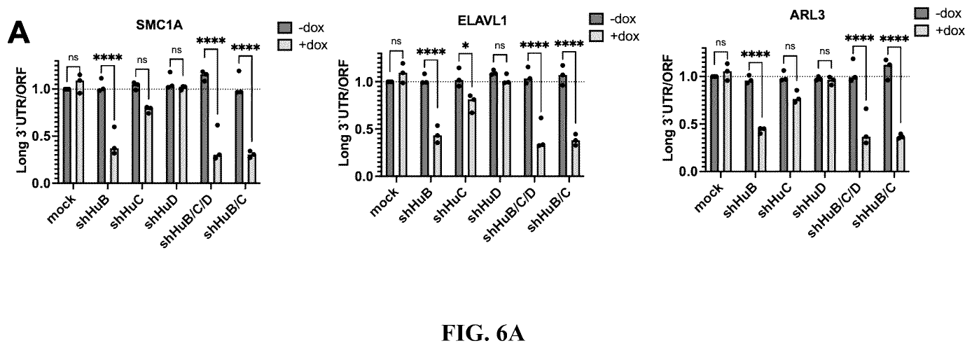

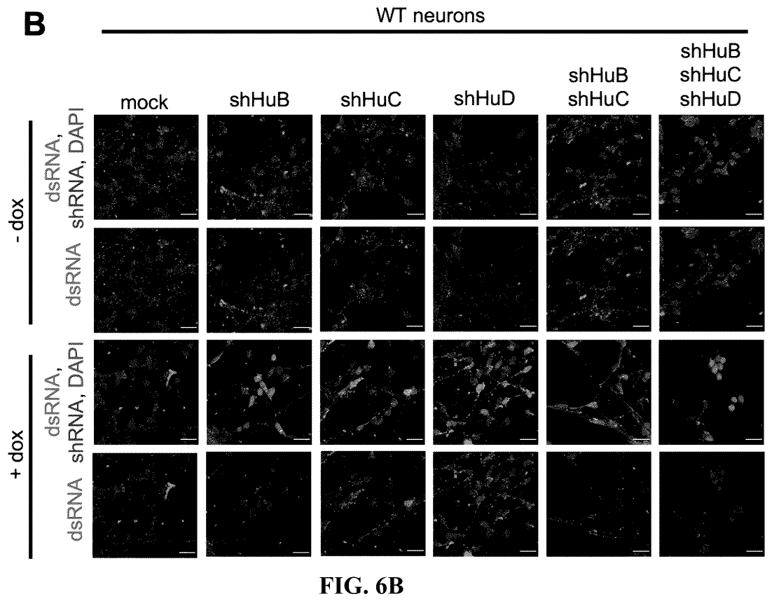

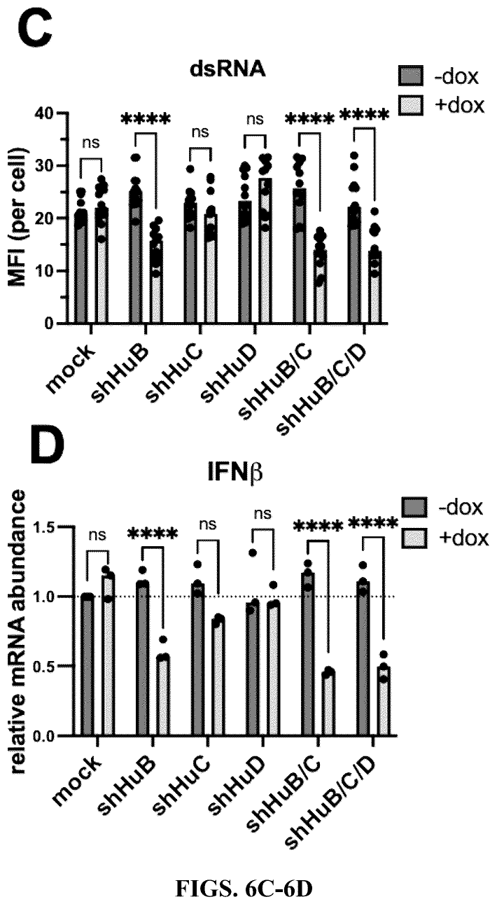

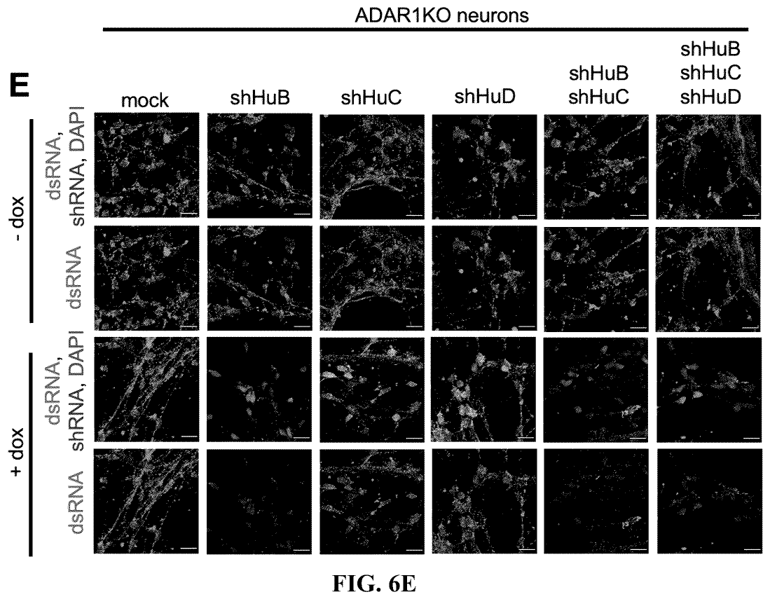

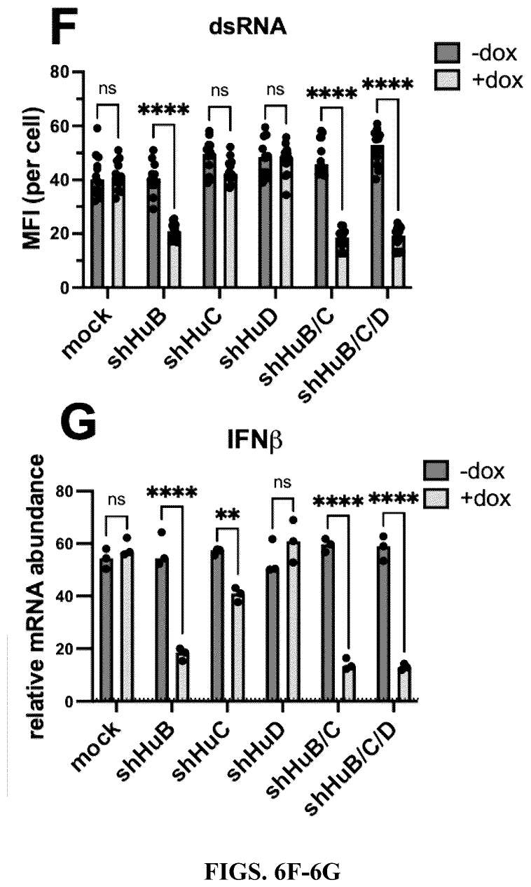

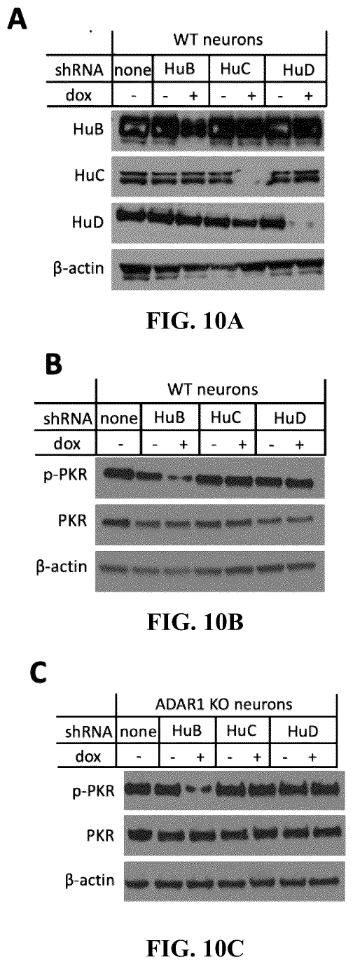

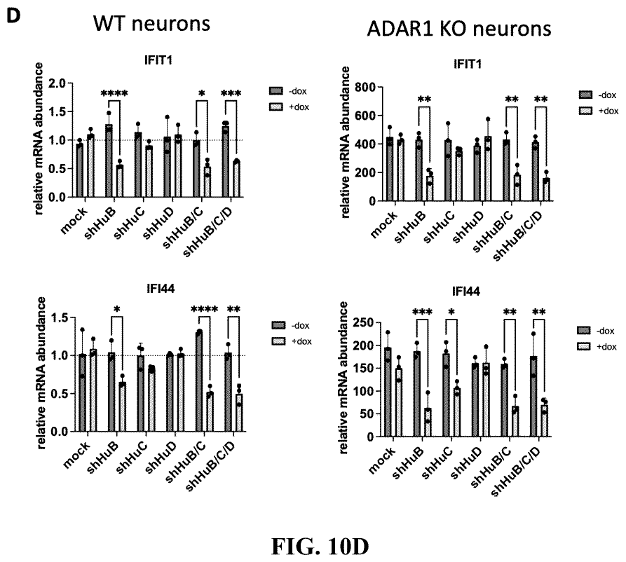

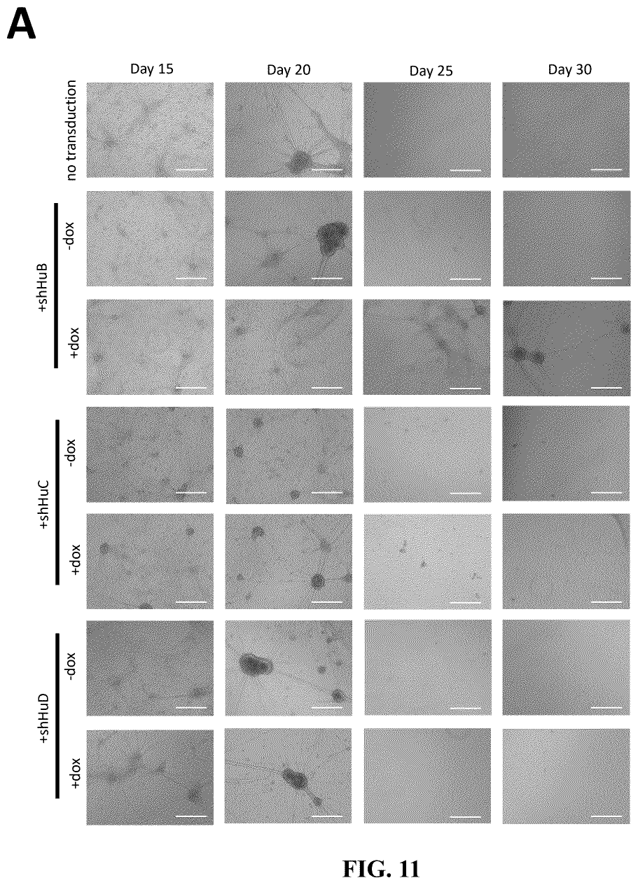

We next sought to further examine the role of HuB, -C, -D proteins in a more physiologically relevant setting. As these proteins are enriched in neurons, we used doxycycline inducible shRNAs to downregulate HuB, HuC, and HuD in day 20 neurons to analyze their function without potentially impacting neuron differentiation. We confirmed efficient knockdown of HuB, -C, and -D in neurons (FIG. 10A). We found that loss of HuB greatly decreased 3′UTR length and loss of HuC showed a slight trend of decreased 3′UTR length, but loss of HuD did not alter 3′UTR length (FIG. 6A). We next investigated if HuB, -C, or -D also maintained dsRNA levels. We show that loss of HuB alone, loss of HuB and HuC together, or loss of all three ELAVL proteins results in a decrease in dsRNA burden in both WT (FIG. 6B, 6C) and ADAR1 KO (FIG. 6E, 6F) neurons. Concordant with reduced dsRNA levels, we also observed reduced IFNβ (FIG. 6D, 6G) and ISG production (FIG. 10D), and PKR activation in both WT (FIG. 10B) and ADAR1 KO (FIG. 10C) neurons. Notably, we observed a striking drop in dsRNA levels in HuB-depleted ADAR1 KO neurons (FIG. 6E, 6F), and downregulating HuB, but not HuC or HuD, prolonged the survival of ADAR1 KO neurons (FIG. 11). In summary, human neurons heavily depend on HuB, and to a lesser extent on HuC, to elongate 3′UTRs, increase dsRNA levels, and induce inflammation as measured by type I IFN and PKR activation.

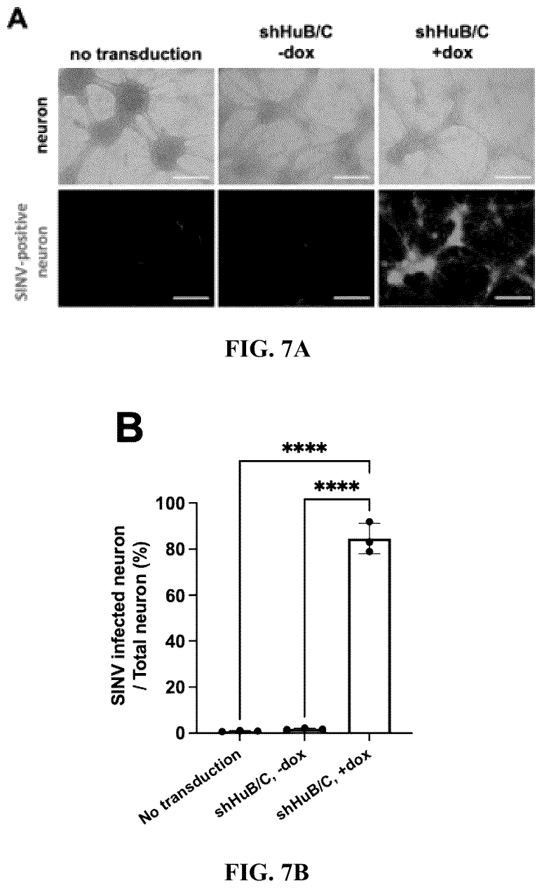

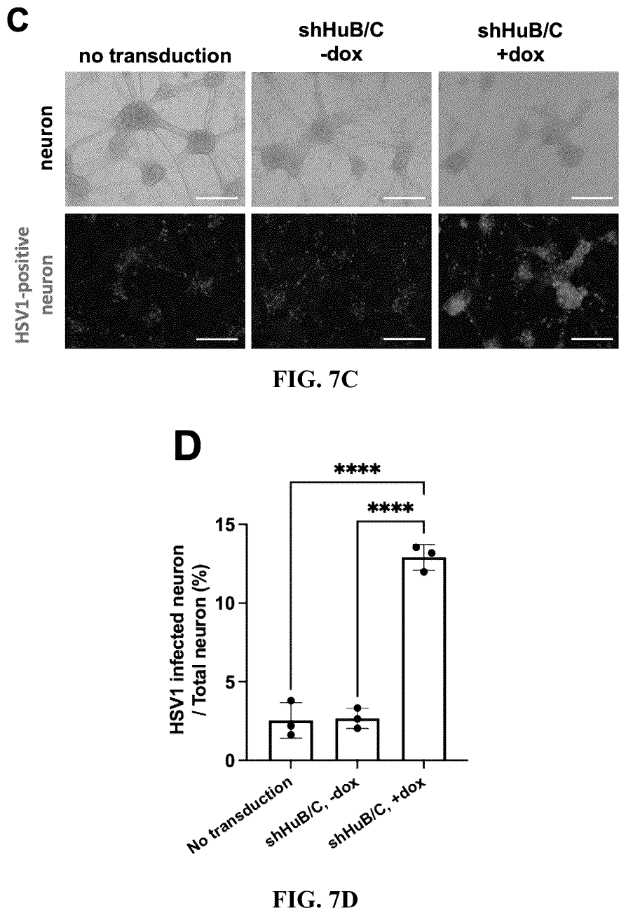

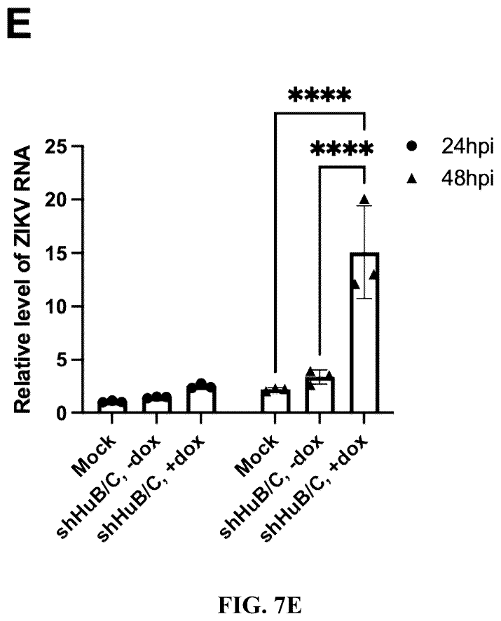

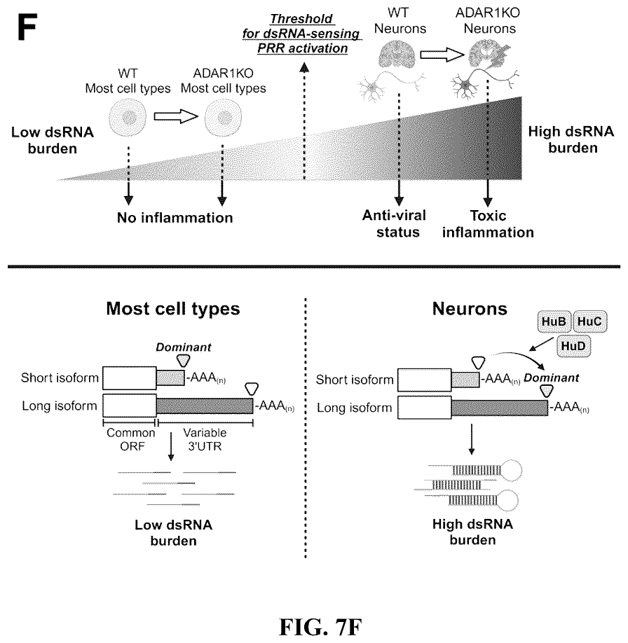

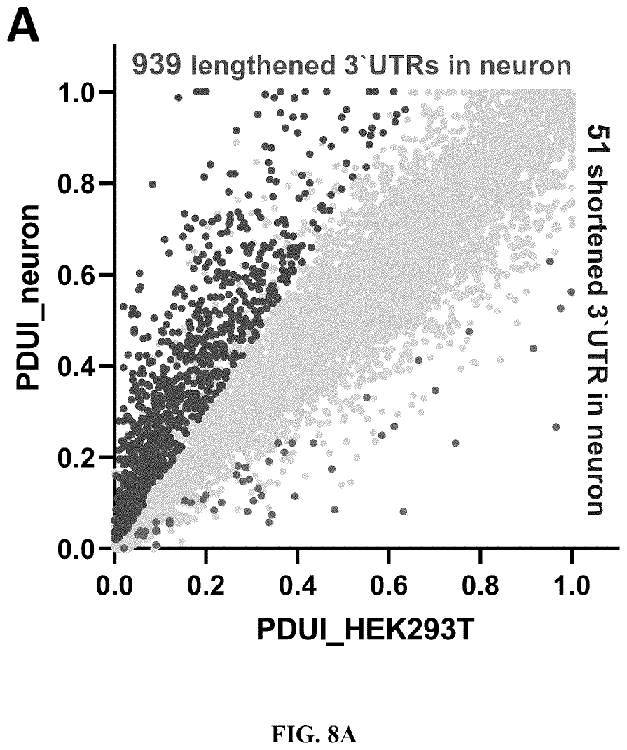

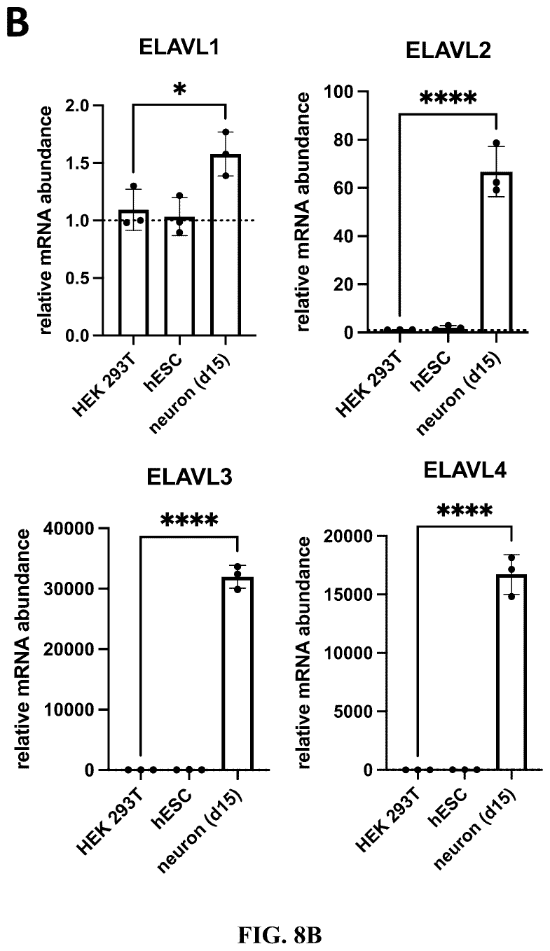

Since, ELAVL proteins, especially HuB and HuC, were required to maintain tonic IFN production and PKR activation in WT neurons, we investigated if these proteins confer resistance to viral infection. To test this, we depleted ELAVL proteins in neurons and measured infectivity with multiple viruses. We depleted both HuB and HuC simultaneously because we observed this resulted in the greatest decrease in dsRNA and IFN production (FIG. 6). Downregulating HuB and HuC in neurons resulted in significantly higher rates of SINV infection (FIG. 7A, 7B).

Next, we examined if HuB/C depletion could also impact the infectivity of neurotropic viruses. Herpes Simplex Virus 1 (HSV-1) is a DNA virus that can cause HSV-1 encephalitis, the most common form of sporadic viral encephalitis in the Western world. Using HSV-1 with a GFP reporter (63), we found that loss of HuB and HuC significantly increased the infectivity of HSV-1 in neurons (FIG. 7C, 7D). Importantly, tonic IFN has been proposed to be vital in preventing HSV-1 infection of neurons (27), and our findings serve to underline the importance of ELAVL proteins in maintaining tonic IFN production to protect from viral infection. In addition to HSV-1, we also tested Zika virus (ZIKV), which can cause severe neurological defects. Depletion of HuB/C significantly increased the infectivity of ZIKV in neurons 48 hours post-infection (FIG. 7E). Taken together, our data suggest that HuB and HuC elongate 3′UTRs, trigger dsRNA-induced immunity, and confers an intrinsic antiviral state to neurons (FIG. 7F).

Effective differentiation of patient-derived iPSCs into neurons: Induced pluripotent stem cells (iPSCs) derived from either healthy control patients or patients with amyotrophic lateral sclerosis (ALS) were differentiated into neural progenitor cells (NPCs) and neurons. Immunofluorescent staining was performed for various markers was completed to confirm cell identity: OCT-4A is a marker of stem cells, nestin is a marker of NPCs, and TUJ1 and MAP2 are both neuronal markers. Patient-derived iPSCs differentiated into neurons.

ALS neurons were found to express higher levels of dsRNA and inflammation than healthy controls: iPSCs from both healthy patients and patients with ALS were differentiated into neurons. iPSCs were stained with the J2 antibody to detect dsRNA levels and quantification of dsRNA levels was performed. Relative IFNβ levels were measured by qPCR. RPS11 was used as a housekeeping gene. Immunoblot was used to measure PKR activation. It was found that ALS neurons were found to express higher levels of dsRNA and inflammation than healthy controls.

Significantly, knockdown of HuB in patient-derived motor neurons was found to decrease dsRNA burden and inflammation: iPSCs from both healthy patients and patients with ALS were differentiated into motor neurons. The motor neurons were transduced with a lentivirus containing doxycycline-inducible shRNA targeting the gene ELAVL2 (HuB) at day 11 post-differentiation. Dox treatment started at day 14, and cells were harvested at day 20. Immunofluorescent staining for various markers was performed, including TUJ1 and MAP2 (neuronal markers), and ISL1 (a specific marker of motor neurons). Immunoblots were performed demonstrating PKR activation and HuB levels. N2 hESCs were used as a comparison. Immunofluorescent staining of dsRNA in motor neurons with HuB knockdown was clear and dsRNA levels were quantified. Relative IFNβ levels were measured by qPCR and RPS11 used as a housekeeping gene. Doxycycline-inducible shRNA targeting the gene ELAVL2 (HuB) significantly decreased dsRNA burden and inflammation in ALS cells.

### DISCUSSION

A common paradigm in innate immunity is that PRRs discriminate between self vs. non-self ligands. However, we show that the neuronal transcriptome is intrinsically enriched for immunostimulatory dsRNAs that are constantly sensed by PRRs, even in homeostasis (FIG. 7F). Hence, the distinction between self vs. non-self dsRNAs may not be as strict as previously believed, especially in neurons. One potential benefit of neuronal dsRNA may be to enhance CNS resistance to viral infections. We demonstrated that depleting dsRNAs in WT neurons dampened tonic type I IFN levels and PKR activation and led to increased susceptibility to infection by SINV, HSV-1, and ZIKV. Hence, the intrinsically high dsRNA levels in neurons may prime the activation of various PRRs (e.g., MDA5, TLR3, PKR etc.) to induce a pre-existing and low-grade immune response that can pre-emptively counteract viral infection. Importantly, this idea is in line with the emerging concept that the brain heavily relies on ‘constitutive immune mechanisms’ for immediate control of infection that would minimize excessive cell death and inflammation that can cause irreversible damage to the brain (27, 64-66). Such pre-emptive defense mechanisms would be particularly important for post-mitotic neurons that have limited capacity to regenerate following cell death or injury.

Pioneering investigations into inborn errors that cause susceptibility to HSV-1 encephalitis have uncovered mutations in various RNA-associated pathways (TLR3 signaling, RNA lariat formation, and snoRNA biogenesis) (27, 64, 65). Additionally, mutations in RNA polymerase III is also associated with severe varicella zoster virus in the CNS (67). How perturbation of RNA homeostasis leads to weakened antiviral defense in the CNS is incompletely understood. Our study sheds new light on the potential mechanistic underpinning of these observations. We speculate that mutations that alter neuronal dsRNA levels or sensing of neuronal dsRNAs by PRRs may result in weakened basal immune activation pre-infection, thereby lowering the threshold for viruses to successfully establish infection.

While neuronal dsRNAs may be beneficial in antiviral defense, we show that high levels of dsRNA in neurons could also pose a risk for harmful neuroinflammation when defective ADAR1 leads to excessively high levels of dsRNA. Elevated type I IFN production in the brain has been reported since the 1980s when AGS was first diagnosed, but why the brain is especially prone to inflammation has been a long-standing question (8, 12, 13). Our study suggests that the inherently high dsRNA level in neurons could provide a molecular basis for why inflammation is most prominent in the AGS brain. We demonstrate that cell types with intrinsically lower dsRNA load tolerated the absence of ADAR1 and subsequent increase in dsRNA levels, but cell types with intrinsically higher dsRNA load such as neurons, could not tolerate the absence of ADAR1 as it led to excessively high dsRNA levels that trigger uncontrolled inflammation (FIG. 7F).

High-dsRNA levels in neurons provides molecular insight into how a prominent IFN signature and chronic PKR activation develops in many neurodegenerative disorders including Alzheimer's disease or especially ALS, where perturbation of RNA binding protein dosage or expanded RNA repeat elements is a major cause of disease (2-7, 68). Additionally, regardless of what initially triggers aberrant type I IFN production in the brain (e.g., viral infection, excessive self-RNA or self-DNA sensing etc.) (69-71), the intrinsically high dsRNA levels in neurons can pose a risk for creating a positive feedback loop for chronic brain inflammation. Type I IFN signaling further elevates PRR levels—PRRs are ISGs—that can lead to increased neuronal dsRNA sensing by PRRs, further amplifying type I IFN production.

3′UTRs are best known to regulate mRNA localization, stability, and translation (51). While the brain is understood to globally lengthen 3′UTRs, the functions of these longer 3′UTRs are incompletely understood (52). We developed a method to force 3′UTR lengthening by ectopically expressing just three neuron-enriched genes ELAVL2, -3, and -4 (HuB, -C, D) in a non-neuronal cell type. With this new experimental tool, we were able to ascribe a previously undescribed function to long 3′UTRs; a rich source for dsRNA that bolsters innate immunity and protects against viral infection. These 3′UTRs may give rise to dsRNA either by incorporating repetitive elements (e.g., Alus) prone to forming dsRNA structures or by forming complementary dsRNA with neighboring genes transcribed in opposite direction (i.e. cis-natural antisense transcripts [cis-NATs]) (72). How ELAVL proteins lengthen 3′UTRs in humans remains unclear. ELAV/Hu proteins in Drosophila are thought to lengthen 3′UTRs by binding near the proximal polyadenylation site, which mediates bypass of the proximal polyadenylation sites and promotes usage of distal polyadenylation sites (57).

From a therapeutic perspective, 3′UTRs can be lengthened to boost immune responses since increasing immunostimulatory self-dsRNA levels is remarkably effective in cancer immunotherapies (73-75), or 3′UTRs can be shortened to reduce immune/inflammatory responses (e.g., neuroinflammatory conditions). Targeting ELAVL2 (HuB) in ADAR1 KO neurons led to shorter 3′UTRs, reduced dsRNA levels and inflammation, and markedly enhanced cell survival of ADAR1 KO neurons. Hence targeting dsRNAs, the upstream trigger for disease, can be an effective therapeutic strategy to treat AGS. shRNA knockdown of HuB in patient-derived motor neurons was found to decrease dsRNA burden and inflammation.

### Materials and Methods

### Study Design

The main objective of this study was to identify a mechanism that rendered neurons more susceptible to inflammation, in cases of both disease and health. We utilized hESCs to generate neurons and to allow for genetic manipulation. We assessed the level of dsRNA in cells using immunofluorescent confocal microscopy, and measured inflammation via qPCR of several markers (IFN and ISGs) and Western blot of PKR activation. We used lentiviral-delivered shRNA to identify genes responsible for both increasing dsRNA levels and inducing the inflammatory response. qPCR and RNA sequencing were used to identify changes in 3′UTR length, and viral infection models were used to identify a functional significance for neural constitutive inflammation.

### Cell Lines

ADAR1 KO HEK-293T cells and ADAR1 KO male HUES8 hESCs, and their corresponding WT control cell lines were generated as described previously (9). Female WA-09 hESCs were obtained from WiCell. WT and ADAR1 KO HeLa cells were generously provided by Roberto Cattaneo (Mayo Clinic). ADAR1/MIDA5 DKO HEK-293T cells were generated using CRISPR-Cas9 and gRNA sequences.

### Cell Culture Reagents

HEK-293T and HeLa cells were cultured in DMEM supplemented with 10% FBS and 1% non-essential amino acids. Both HUES8 and WA-09 hESCs were cultured in mTESR1 with its supplement (Stem Cell Technologies) and were split using ReLeSR (Stem Cell Technologies).

For treatment with various polymerase inhibitors, cells were treated for 12 hours prior to harvest with either ActD (10 ug/mL, Sigma), α-amanitin (25 ug/mL, Sigma), or IMT1 (1 uM, MedChemExpress).

To knockdown various immune proteins in neurons, lentiviruses were generated using a pTRIPZ vector (Dharmacon) that carried shRNA. At day 11 post differentiation, neurons were transduced with lentivirus carrying doxycycline inducible shRNA targeting MDA5, RIG-I, MAVS, STING, MyD88, TRIF, PKR, HuB, HuC, and HuD. Doxycycline treatment started at day 14 post differentiation.

Ectopic expression of HuB, HuC, and HuD was done using the pFRT backbone (Addgene ID 26360) expressing ELAVL2, ELAVL3, and ELAVL4 respectively (Addgene ID: 65758, 65759, 65760). HEK-293T cells were transfected using Lipofectamine 2000 with either 100 ng of a single plasmid, or 33.3 ng each of all three plasmids in combination. Transfections lasted for 48 hours before cells were harvested for analysis.

### NPC Differentiation Protocol

hESCs were differentiated into NPCs using an adherent monolayer culture system (Stem Cell Technologies, STEMdiffr™ SMADi Neural Induction Kit). hESCs were plated into matrigel (Corning) coated wells and grown with neural induction media for approximately 15 days. This was followed by maintenance and expansion in neural progenitor media. Importantly, compared to previous work (9), we increased the density of hESCs at plating (3*10⁵cells/cm2) to obtain a more homogenous and synchronized NPC population.

### Neuron Differentiation Protocol

NPCs were differentiated into neurons using an adherent monolayer culture system (Stem Cell Technologies, BrainPhys™ Neuronal Medium N2-A & SM1 Kit) (21). NPCs were plated into wells coated with poly-L-ornithine and laminin and grown in neuronal media. Media was changed every 3 days and cells were harvested at the timepoints indicated.

### Hepatocyte-Like Cell (HLC) Differentiation Protocol

HLCs were generated as described previously (22). Briefly, hESCs were dissociated using Gentle Cell Dissociation Reagent (Stem Cell Technologies) and plated in matrigel coated wells. Endoderm differentiation was accomplished using the STEMdiff Definitive Endoderm kit (Stem Cell Technologies). Endoderm cells were re-plated in matrigel coated wells and HLC differentiation was started by treating with stage 1 hepatic differentiation media for 8 days (DMEM/F-12 supplemented with 10% KOSR, 1% non-essential amino acids, 0.8% pen-strep, 1% glutamine, 100 ng/mL HGF [Peprotech]), and 1% DMSO). On day 8, stage 2 hepatic differentiation media (replacing the DMSO in stage 1 media with 0.1 uM dexamethasone [Sigma]). At day 11, Hepatocyte Culture Media (HCM Lonza) was added, supplemented with 20 ng/mL of oncostatin-M (R&D Systems). Cells were maintained in HCM until harvested at the indicated times (days post-differentiation from endoderm).

### Cardiomyocyte Differentiation Protocol

hESCs were differentiated into cardiomyocytes using the STEMdiff Ventricular Cardiomyocyte Differentiation kit (Stem Cell Technologies). The hESCs were dissociated using Gentle Cell Dissociation Reagent and plated into matrigel coated wells. Cardiomyocyte differentiation media was added supplemented with matrigel. Differentiation media was changed every 2 days for 6 days, and on day 8 cardiomyocyte maintenance media was added. Maintenance media was changed every 2 days until the cells were harvested at day 24.

### Motor Neuron Differentiation Protocol

Motor neurons were generated using an embryoid body-mediated protocol adapted from a previous study (23). hESCs were dissociated using accutase to generate a single cell suspension and were plated in low adherence culture dishes in N2B27 media (50% DMEM/F-12 and 50% neurobasal medium [Thermo Fisher Scientific], supplemented with N2, B27, 1% pen-strep, 1% Glutamax [all Thermo Fisher Scientific], and 0.1% β-mercaptoethanol [Sigma]), further supplemented with ascorbic acid (Sigma), FGF2 (Thermo Fisher Scientific), Y-27632 (abcam), SB431542 (Sigma), LDN 193189 (Tocris), and Chir-99021 (Tocris). At day 2 post plating, ascorbic acid, SB431542, LDN 193189, and Chir-99021 were maintained and retinoic acid (Sigma) and smoothened agonist (SAG, Sigma) were added. At day 6, the media was changed, including only ascorbic acid, retinoic acid, and SAG. At day 9, in addition to the day 7 supplements, DAPT (Sigma) was added. At day 11 BDNF (R&D Systems) was added, and at day 14 GDNF (R&D Systems) was added. On day 16, the embryoid bodies were dissociated using accumax and plated into a monolayer in wells coated with poly-L-ornithine and laminin.

Monolayers of motor neurons were maintained in neurobasal media supplemented with: 1% Glutamax, 1% non-essential amino acids, 0.1% β-mercaptoethanol, N2, B27, BDNF, GDNF, CNTF (R&D Systems), and 1 uM of 1:1 uridine and fluorodeoxyuridine (Sigma). Motor neurons were harvested at the indicated time points (days post-dissociation)

### Immunofluorescent Microscopy

Cells grown on glass coverslips were washed with PBS and fixed using 4% paraformaldehyde for 20 minutes at 4° C. Samples were then blocked and permeabilized in PBTG (PBS, 1% BSA, 0.1% Triton-X100, 10% goat serum) for 1 hour at room temperature. Primary antibodies were diluted in PBTG and stained overnight at 4° C. Secondary antibodies (either goat anti-mouse IgG Alexa Fluor 488 or goat anti-rabbit IgG Alexa Fluor 594, Invitrogen (A11001 and A11012)) and DAPI were diluted in PBTG and stained for 1 hour at room temperature prior to mounting. Samples were imaged on an LSM-710 confocal microscope.

The following primary antibodies were used: OCT-4A (1:2000, Cell Signaling Technology C52G3), nestin (1:200, Neuromics M022183), TUJ1 (1:1000, Biolegend 801201), MAP2 (1:100, Cell Signaling Technology 4542S), FoxA2 (1:400, Cell Signaling Technology 8186P), HNF4-α (1:500, Cell Signaling Technology 3113S), albumin (1:500, Cedarlane CL2513A), ISL1 (1:100, DSHB 39.4D5), cardiac troponin T (1:100, Thermofisher MA5-12960), J2 (1:300, ExAlpha10010500) (14), K1 (1:300, ExAlpha 10020200), and 9D5 (1:250, Absolute Antibody 00458-23.0). RNASelect stain was obtained from Thermo Fisher Scientific.

For samples treated with nucleases, after fixation there was a permeabilization step in 0.1% Triton-X100 for 1 hour. After, samples were incubated for 30 minutes at 37° C. with either ssRNase (RNase T1, 100 U/mL, Sigma), dsRNase (RNase III, 40 U/mL, Sigma), or DNase (RQ1, 40 U/mL, Promega) diluted in PBS containing 5 mM magnesium chloride. Samples were then stained as described above.

polya Fish

To image polyadenylated RNA, 30-nucleotide long polyT probes were conjugated to Cy5 (Integrated DNA Technologies). Some samples were treated with α-amanitin as shown above. After differentiation to appropriate cell types, cells were washed with PBS and fixed using 4% paraformaldehyde for 10 minutes. The cells were then permeabilized in 70% ethanol overnight at −20° C. Samples were washed and hybridized overnight at 37° C. in the following buffer: 125 nM probe, 2×SSC, 20% formamide, 0.1 g/mL dextran sulfate, 1 mg/mL E. coli tRNA, 2 mM vanadyl ribonucleoside complex, and 0.1% Tween20. The samples were washed with 2X SCC and 20% formamide, counterstained with DAPI, and imaged as described above.

### Tissue Staining

Frozen sagittal brain sections from healthy C57BL/6 mice were obtained from Zyagen. Heart and skin tissue from healthy C57BL/6 mice were embedded in OCT and sectioned. All tissue sections were fixed in 4% paraformaldehyde for 20 minutes at 4° C., and permeabilized and blocked in PBTG. Primary antibodies (9D5 from Absolute Antibody 00458-23.0), and NeuN from abcam ab134014) were stained as described above. Secondary antibodies (either goat anti-chicken IgG Alexa Fluor 488 or goat anti-rabbit IgG Alexa Fluor 594, Invitrogen (11039 and 11012) and DAPI were stained as described above. Samples were imaged on an LSM-710 confocal microscope.

Mitochondria and dsRNA Co-Staining

Cells were treated with Mitotracker Deep Red (Invitrogen) for 1 hour before fixation. Subsequently, the cells were stained with the J2 antibody, as described above. Samples were imaged on an LSM-710 confocal microscope. The proportion of colocalization area was analyzed by ImageJ.

### Image Analysis

Images were processed and analyzed using ImageJ. MFI of individual cells was determined by tracing cell outlines. MFI data depicts 12 individual cells taken from at least 3 independent images. For colocalization analysis, the JACop plugin was used to measure MFI of 4 independent images.

qRT-PCR (qPCR)

RNA was extracted directly from cell samples using the Direct-zol RNA Miniprep kit (Zymo Research). cDNA conversion was completed using the ProtoScript II First Strand cDNA Synthesis kit (New England Biolabs). PowerUp SYBR Green (Thermo Fisher Scientific) and QuantStudio 3 Real-Time PCR System were used for qRT-PCR, and all primers are listed in Table S1. RPS11 or 18S rRNA were used as a housekeeping gene for normalization.

### Western Blot

Cell samples were lysed directly using NuPAGE LDS Sample Buffer (Thermo Fisher Scientific) and 400 mM DTT (Sigma). Lysates were homogenized by passing through a 26G needle and were denatured by boiling for 10 minutes. The protein samples were run on NuPAGE 4-12% Bis-Tris Protein Gels (Thermo Fisher Scientific) and were blotted on to nitrocellulose membrane. The following proteins were probed: MDA5 (Cell Signaling Technology 5321), RIG-I (Cell Signaling Technology 3743S), MAVS (Cell Signaling Technology 3993S), STING (Cell Signaling Technology 13647S), MyD88 (Cell Signaling Technology 4283S), TRIF (Cell Signaling Technology 4596S), p-PKR (phospho T451, abcam 227983), PKR (abcam 32506), p-IRF3 (phospho S386, abcam 76493), IRF3 (Cell Signaling Technology 11904), ADAR1 (Cell Signaling Technology 81284S), ADAR2 (Atlas Antibodies 018277), FLAG (Santa Cruz 166355), and -actin (Sigma A3854). Blots were developed using a chemiluminescent (film) method.

### RNA Extraction and Library Preparation

For the HuB/C/D ectopic expression experiment (biological triplicates of HEK-293T_Mock, HEK-293T_EV, and HEK-293T_HuB/C/D), RNA was extracted with the Direct-zol RNA MiniPrep (Zymo Research). ERCC RNA Spike-In Mix (Thermo Fisher Scientific) was added to each RNA sample. rRNA was depleted using the RiboMinus™ Eukaryote Kit (Thermo Fisher Scientific). Libraries were prepared with the NEBNext® Ultra™ II Directional RNA Library Prep Kit (New England Biolabs). The 9 libraries were pooled and sequenced on the Illumina Novaseq platform with a read length of 150 nt in the paired end configuration. For analyzing global 3′UTR extension in neurons, biological triplicates of RNA samples from neurons and HEK 293T were extracted with the Direct-zol RNA MiniPrep (Zymo Research). After adding ERCC RNA Spike-In Mix (Thermo Fisher Scientific), libraries were prepared with the TruSeq stranded Total RNA with Ribozero kit (Illumina). The libraries were sequenced on the Illumina Novaseq platform with a read length of 100 nt in paired end configuration.

### Virus Infection

### Viruses and Infections

SINV, HSV-1 (63), and ZIKA viruses originated from TE5′2J, KOS-1, and MR766 strains, respectively. SINV possesses a dual reporter system that produces a blue fluorescent protein (BFP) and a green fluorescent protein (GFP). HSV-lexpresses GFP. Following virus infection of cells at the appropriate multiplicity of infection (MOI), samples were collected for analysis at 6, 24, or 48 hours post-infection. Fluorescent images were acquired using the EVOS M5000 microscope system (Invitrogen).

### Flow Cytometry

Cells were detached with accumax (Stem Cell Technologies) and fixed using 2% paraformaldehyde for 20 minutes at 4° C. Samples were resuspended in 3% FBS solution and analyzed using a BD LSR Fortessa flow cytometer (BD Biosciences).

### RNA Sequencing Data Analysis

### Preprocessing and Alignment

After quality control (FastQC v0.11.5), adaptor and low-quality reads were trimmed from FASTQ files using fastp v0.20.1, and duplicated reads were removed using fastuniq v1.1. Preprocessed reads were mapped to the human genome (hg38) using STAR v2.7.3a to generate BAM files.

### Alternative Polyadenylation Site Analysis

Uniquely mapped reads in BAM files were converted to bedgraph files using bedtools v2.30.0. DaPars v2.1 took the bedgraph files as input, inferred alternative polyadenylation sites, and provided a percentage of distal polyadenylated usage index (PDUI) (56). We only used genes for which at least two PDUI values were measured in each sample for statistical analysis. Significant 3′UTR changes were defined as alterations with false discovery rate (FDR)≤0.05 and |log 2(Fold Change of PDUI)|≥0.59.

### Gene Expression Analysis

Read counts were calculated using featureCounts v1.6.4 with a GENCODE v38 primary assembly annotation file. Genes with a raw count of less than 10 in all sample conditions were excluded from subsequent analysis. Normalization (using the TMM method) and identification of differentially expressed genes was performed with the edgeR package in Bioconductor (78). Genes with a false discovery rate (FDR)≤0.001 and |log 2(Fold Change of CPM)|≥1.585 are annotated as differentially expressed genes (DEG). Gene ontology (Biological process) enrichment of upregulated genes was performed with the clusterProfiler Bioconductor package (79).

### Human Protein Atlas Data

The tissue gene expression data was sourced from the Human Protein Atlas database, and the expression levels of Type I IFN (IFNβ and IFNα subtypes) were extracted. The resultant dataset was visualized using the heatmap function in R.

### Statistical Analysis

Except for RNA sequencing analysis, all statistical analysis was performed using Graphpad Prism 9 software. Where indicated, a one- or two-way ANOVA with multiple comparisons was performed with Tukey post-hoc correction.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### REFERENCES

1. Y. G. Chen, S. Hur, Cellular origins of dsRNA, their recognition and consequences. Nat. Rev. Mol. Cell Biol. 23 (2022), doi:10.1038/s41580-021-00430-1.
2. J. Hugon, F. Mouton-Liger, J. Dumurgier, C. Paquet, PKR involvement in Alzheimer's disease. Alzheimers Res Ther. 9, 83 (2017).
3. T. Zu, S. Guo, O. Bardhi, D. A. Ryskamp, J. Li, S. K. Tusi, A. Engelbrecht, K. Klippel, P. Chakrabarty, L. Nguyen, T. E. Golde, N. Sonenberg, L. P. W. Ranum, Metformin inhibits RAN translation through PKR pathway and mitigates disease in C9orf72 ALS/FTD mice. Proc. Natl. Acad. Sci. U.S.A 117 (2020), doi:10.1073/pnas.2005748117.
4. S. Rodriguez, A. Sahin, B. R. Schrank, H. Al-Lawati, I. Costantino, E. Benz, D. Fard, A. D. Albers, L. Cao, A. C. Gomez, K. Evans, E. Ratti, M. Cudkowicz, M. P. Frosch, M. Talkowski, P. K. Sorger, B. T. Hyman, M. W. Albers, Genome-encoded cytoplasmic double-stranded RNAs, found in C90RF72 ALS-FTD brain, propagate neuronal loss. Sci. Transl. Med. 13 (2021), doi:10.1126/scitranslmed.aaz4699.
5. E. R. Roy, G. Chiu, S. Li, N. E. Propson, R. Kanchi, B. Wang, C. Coarfa, H. Zheng, W. Cao, Concerted type I interferon signaling in microglia and neural cells promotes memory impairment associated with amyloid β plaques. Immunity. 55, 879-894.e6 (2022).
6. H. Lee, R. J. Fenster, S. S. Pineda, W. S. Gibbs, S. Mohammadi, J. Davila-Velderrain, F. J. Garcia, M. Therrien, H. S. Novis, F. Gao, H. Wilkinson, T. Vogt, M. Kellis, M. J. LaVoie, M. Heiman, Cell Type-Specific Transcriptomics Reveals that Mutant Huntingtin Leads to Mitochondrial RNA Release and Neuronal Innate Immune Activation. Neuron (2020), doi:10.1016/j.neuron.2020.06.021.
7. E. Ochoa, P. Ramirez, E. Gonzalez, J. De Mange, W. J. Ray, K. F. Bieniek, B. Frost, Pathogenic tau-induced transposable element-derived dsRNA drives neuroinflammation. Sci. Adv. 9 (2023), doi:10.1126/sciadv.abg5423.
8. Y. J. Crow, D. B. Stetson, The type I interferonopathies: 10 years on. Nat. Rev. Immunol. 22 (2022), doi:10.1038/s41577-021-00633-9.
9. H. Chung, J. J. A. Calis, X. Wu, T. Sun, Y. Yu, S. L. Sarbanes, V. L. Dao Thi, A. R. Shilvock, H. H. Hoffmann, B. R. Rosenberg, C. M. Rice, Human ADAR1 Prevents Endogenous RNA from Triggering Translational Shutdown. Cell. 172, 811-824 e14 (2018).
10. S. Ahmad, X. Mu, F. Yang, E. Greenwald, J. W. Park, E. Jacob, C. Z. Zhang, S. Hur, Breaching Self-Tolerance to Alu Duplex RNA Underlies MDA5-Mediated Inflammation. Cell. 172, 797-810 e13 (2018).
11. S. Maharana, S. Kretschmer, S. Hunger, X. Yan, D. Kuster, S. Traikov, T. Zillinger, M. Gentzel, S. Elangovan, P. Dasgupta, N. Chappidi, N. Lucas, K. I. Maser, H. Maatz, A. Rapp, V. Marchand, Y. T. Chang, Y. Motorin, N. Hubner, G. Hartmann, A. A. Hyman, S. Alberti, M. A. Lee-Kirsch, SAMHD1 controls innate immunity by regulating condensation of immunogenic self RNA. Mol. Cell. 82 (2022), doi:10.1016/j.molcel.2022.08.031.
12. L. Lodi, I. Melki, V. Bondet, L. Seabra, G. I. Rice, E. Carter, A. Lepelley, M. J. Martin-Niclós, B. Al Adba, B. Bader-Meunier, M. Barth, T. Blauwblomme, C. Bodemer, O. Boespflug-Tanguy, R. C. Dale, I. Desguerre, C. Ducrocq, F. Dulieu, C. Dumaine, P. Ellul, A. Hadchouel, V. Hentgen, M. Hié, M. Hully, E. Jeziorski, R. Lévy, F. Mochel, S. Orcesi, S. Passemard, M. Pouletty, P. Quartier, F. Renaldo, R. Seidl, J. Shetty, B. Neven, S. Blanche, D. Duffy, Y. J. Crow, M. L. Frémond, Differential Expression of Interferon-Alpha Protein Provides Clues to Tissue Specificity Across Type I Interferonopathies. J. Clin. Immunol. 41 (2021), doi:10.1007/s10875-020-00952-x.
13. P. Lebon, J. Badoual, G. Ponsot, F. Goutières, F. Hémeury-Cukier, J. Aicardi, Intrathecal synthesis of interferon-alpha in infants with progressive familial encephalopathy. J. Neurol. Sci. 84 (1988), doi:10.1016/0022-510X(88)90125-6.
14. J. Schonborn, J. Oberstrafβ, E. Breyel, J. Tittgen, J. Schumacher, N. Lukacs, Monoclonal antibodies to double-stranded RNA as probes of RNA structure in crude nucleic acid extracts. Nucleic Acids Res. 19 (1991), doi:10.1093/nar/19.11.2993.
15. Y. Kim, J. Park, S. Kim, M. Kim, M. G. Kang, C. Kwak, M. Kang, B. Kim, H. W. Rhee, V. N. Kim, PKR Senses Nuclear and Mitochondrial Signals by Interacting with Endogenous Double-Stranded RNAs. Mol Cell. 71, 1051-1063 e6 (2018).
16. A. Dhir, S. Dhir, L. S. Borowski, L. Jimenez, M. Teitell, A. Rötig, Y. J. Crow, G. I. Rice, D. Duffy, C. Tamby, T. Nojima, A. Munnich, M. Schiff, C. R. de Almeida, J. Rehwinkel, A. Dziembowski, R. J. Szczesny, N. J. Proudfoot, Mitochondrial double-stranded RNA triggers antiviral signalling in humans. Nature. 560 (2018), doi:10.1038/s41586-018-0363-0.
17. Y. Gao, R. Vasic, Y. Song, R. Teng, C. Liu, R. Gbyli, G. Biancon, R. Nelakanti, K. Lobben, E. Kudo, W. Liu, A. Ardasheva, X. Fu, X. Wang, P. Joshi, V. Lee, B. Dura, G. Viero, A. Iwasaki, R. Fan, A. Xiao, R. A. Flavell, H. B. Li, T. Tebaldi, S. Halene, m6A Modification Prevents Formation of Endogenous Double-Stranded RNAs and Deleterious Innate Immune Responses during Hematopoietic Development. Immunity. 52 (2020), doi:10.1016/j.immuni.2020.05.003.
18. D. P. Reich, B. L. Bass, Mapping the dsrna world. Cold Spring Harb. Perspect. Biol. 11 (2019), doi:10.1101/cshperspect.a035352.
19. M. G. Blango, B. L. Bass, Identification of the long, edited dsRNAome of LPS-stimulated immune cells. Genome Res. 26 (2016), doi:10.1101/gr.203992.116.
20. S. M. Chambers, C. A. Fasano, E. P. Papapetrou, M. Tomishima, M. Sadelain, L. Studer, Highly efficient neural conversion of human ES and iPS cells by dual inhibition of SMAD signaling. Nat Biotechnol. 27, 275-280 (2009).
21. C. Bardy, M. Van Den Hurk, T. Eames, C. Marchand, R. V. Hernandez, M. Kellogg, M. Gorris, B. Galet, V. Palomares, J. Brown, A. G. Bang, J. Mertens, L. Böhnke, L. Boyer, S. Simon, F. H. Gage, Neuronal medium that supports basic synaptic functions and activity of human neurons in vitro. Proc. Natl. Acad. Sci. U.S.A 112 (2015), doi:10.1073/pnas.1504393112.
22. X. Wu, V. L. Dao Thi, Y. Huang, E. Billerbeck, D. Saha, H. H. Hoffmann, Y. Wang, L. A. V Silva, S. Sarbanes, T. Sun, L. Andrus, Y. Yu, C. Quirk, M. Li, M. R. MacDonald, W. M. Schneider, X. An, B. R. Rosenberg, C. M. Rice, Intrinsic Immunity Shapes Viral Resistance of Stem Cells. Cell (2017), doi:10.1016/j.cell.2017.11.018.
23. Y. Maury, J. Côme, R. A. Piskorowski, N. Salah-Mohellibi, V. Chevaleyre, M. Peschanski, C. Martinat, S. Nedelec, Combinatorial analysis of developmental cues efficiently converts human pluripotent stem cells into multiple neuronal subtypes. Nat. Biotechnol. 33 (2015), doi:10.1038/nbt.3049.
24. Y. E. Lewis, A. Moskovitz, M. Mutlak, J. Heineke, L. H. Caspi, I. Kehat, Localization of transcripts, translation, and degradation for spatiotemporal sarcomere maintenance. J. Mol. Cell. Cardiol. 116 (2018), doi:10.1016/j.yjmcc.2018.01.012.
25. N. A. Bonekamp, B. Peter, H. S. Hillen, A. Felser, T. Bergbrede, A. Choidas, M. Horn, A. Unger, R. Di Lucrezia, I. Atanassov, X. Li, U. Koch, S. Menninger, J. Boros, P. Habenberger, P. Giavalisco, P. Cramer, M. S. Denzel, P. Nussbaumer, B. Klebl, M. Falkenberg, C. M. Gustafsson, N. G. Larsson, Small-molecule inhibitors of human mitochondrial DNA transcription. Nature. 588 (2020), doi:10.1038/s41586-020-03048-z.
26. D. J. Gough, N. L. Messina, C. J. Clarke, R. W. Johnstone, D. E. Levy, Constitutive type I interferon modulates homeostatic balance through tonic signaling. Immunity. 36, 166-174 (2012).
27. D. Gao, M. J. Ciancanelli, P. Zhang, O. Harschnitz, V. Bondet, M. Hasek, J. Chen, X. Mu, Y. Itan, A. Cobat, V. Sancho-Shimizu, B. Bigio, L. Lorenzo, G. Ciceri, J. McAlpine, E. Anguiano, E. Jouanguy, D. Chaussabel, I. Meyts, M. S. Diamond, L. Abel, S. Hur, G. A. Smith, L. Notarangelo, D. Duffy, L. Studer, J. L. Casanova, S. Y. Zhang, TLR3 controls constitutive IFN-β antiviral immunity in human fibroblasts and cortical neurons. J. Clin. Invest. 131 (2021), doi:10.1172/JCI134529.
28. P. Ejlerskov, J. G. Hultberg, J. Wang, R. Carlsson, M. Ambjorn, M. Kuss, Y. Liu, G. Porcu, K. Kolkova, C. Friis Rundsten, K. Ruscher, B. Pakkenberg, T. Goldmann, D. Loreth, M. Prinz, D. C. Rubinsztein, S. Issazadeh-Navikas, Lack of Neuronal IFN-beta-IFNAR Causes Lewy Body- and Parkinson's Disease-like Dementia. Cell. 163, 324-339 (2015).
29. S. Hosseini, K. Michaelsen-Preusse, G. Grigoryan, C. Chhatbar, U. Kalinke, M. Korte, Type I Interferon Receptor Signaling in Astrocytes Regulates Hippocampal Synaptic Plasticity and Cognitive Function of the Healthy CNS. Cell Rep. 31 (2020), doi:10.1016/j.celrep.2020.107666.
30. R. Medzhitov, The spectrum of inflammatory responses. Science (80-.). 374 (2021), doi:10.1126/SCIENCE.ABI5200.
31. J. Lonsdale, J. Thomas, M. Salvatore, R. Phillips, E. Lo, S. Shad, R. Hasz, G. Walters, F. Garcia, N. Young, B. Foster, M. Moser, E. Karasik, B. Gillard, K. Ramsey, S. Sullivan, J. Bridge, H. Magazine, J. Syron, J. Fleming, L. Siminoff, H. Traino, M. Mosavel, L. Barker, S. Jewell, D. Rohrer, D. Maxim, D. Filkins, P. Harbach, E. Cortadillo, B. Berghuis, L. Turner, E. Hudson, K. Feenstra, L. Sobin, J. Robb, P. Branton, G. Korzeniewski, C. Shive, D. Tabor, L. Qi, K. Groch, S. Nampally, S. Buia, A. Zimmerman, A. Smith, R. Burges, K. Robinson, K. Valentino, D. Bradbury, M. Cosentino, N. Diaz-Mayoral, M. Kennedy, T. Engel, P. Williams, K. Erickson, K. Ardlie, W. Winckler, G. Getz, D. DeLuca, Daniel MacArthur, M. Kellis, A. Thomson, T. Young, E. Gelfand, M. Donovan, Y. Meng, G. Grant, D. Mash, Y. Marcus, M. Basile, J. Liu, J. Zhu, Z. Tu, N. J. Cox, D. L. Nicolae, E. R. Gamazon, H. K. Im, A. Konkashbaev, J. Pritchard, M. Stevens, T. Flutre, X. Wen, E. T. Dermitzakis, T. Lappalainen, R. Guigo, J. Monlong, M. Sammeth, D. Koller, A. Battle, S. Mostafavi, M. McCarthy, M. Rivas, J. Maller, I. Rusyn, A. Nobel, F. Wright, A. Shabalin, M. Feolo, N. Sharopova, A. Sturcke, J. Paschal, J. M. Anderson, E. L. Wilder, L. K. Derr, E. D. Green, J. P. Struewing, G. Temple, S. Volpi, J. T. Boyer, E. J. Thomson, M. S. Guyer, C. Ng, A. Abdallah, D. Colantuoni, T. R. Insel, S. E. Koester, A Roger Little, P. K. Bender, T. Lehner, Y. Yao, C. C. Compton, J. B. Vaught, S. Sawyer, N. C. Lockhart, J. Demchok, H. F. Moore, The Genotype-Tissue Expression (GTEx) project. Nat. Genet. (2013), doi:10.1038/ng.2653.
32. M. Uhlén, L. Fagerberg, B. M. Hallström, C. Lindskog, P. Oksvold, A. Mardinoglu, Å. Sivertsson, C. Kampf, E. Sjöstedt, A. Asplund, I. M. Olsson, K. Edlund, E. Lundberg, S. Navani, C. A. K. Szigyarto, J. Odeberg, D. Djureinovic, J. O. Takanen, S. Hober, T. Alm, P. H. Edqvist, H. Berling, H. Tegel, J. Mulder, J. Rockberg, P. Nilsson, J. M. Schwenk, M. Hamsten, K. Von Feilitzen, M. Forsberg, L. Persson, F. Johansson, M. Zwahlen, G. Von Heijne, J. Nielsen, F. Pontén, Tissue-based map of the human proteome. Science (80-.). 347 (2015), doi:10.1126/science.1260419.
33. X. Tan, L. Sun, J. Chen, Z. J. Chen, Detection of Microbial Infections Through Innate Immune Sensing of Nucleic Acids. Annu. Rev. Microbiol. (2018), doi:10.1146/annurev-micro-102215-095605.
34. G. I. Rice, P. R. Kasher, G. M. Forte, N. M. Mannion, S. M. Greenwood, M. Szynkiewicz, J. E. Dickerson, S. S. Bhaskar, M. Zampini, T. A. Briggs, E. M. Jenkinson, C. A. Bacino, R. Battini, E. Bertini, P. A. Brogan, L. A. Brueton, M. Carpanelli, C. De Laet, P. de Lonlay, M. del Toro, I. Desguerre, E. Fazzi, A. Garcia-Cazorla, A. Heiberg, M. Kawaguchi, R. Kumar, J. P. Lin, C. M. Lourenco, A. M. Male, W. Marques Jr., C. Mignot, I. Olivieri, S. Orcesi, P. Prabhakar, M. Rasmussen, R. A. Robinson, F. Rozenberg, J. L. Schmidt, K. Steindl, T. Y. Tan, W. G. van der Merwe, A. Vanderver, G. Vassallo, E. L. Wakeling, E. Wassmer, E. Whittaker, J. H. Livingston, P. Lebon, T. Suzuki, P. J. McLaughlin, L. P. Keegan, M. A. O'Connell, S. C. Lovell, Y. J. Crow, Mutations in ADAR1 cause Aicardi-Goutieres syndrome associated with a type I interferon signature. Nat Genet. 44, 1243-1248 (2012).
35. B. L. Bass, H. Weintraub, An unwinding activity that covalently modifies its double-stranded RNA substrate. Cell. 55, 1089-1098 (1988).
36. N. M. Mannion, S. M. Greenwood, R. Young, S. Cox, J. Brindle, D. Read, C. Nellaker, C. Vesely, C. P. Ponting, P. J. McLaughlin, M. F. Jantsch, J. Dorin, I. R. Adams, A. D. Scadden, M. Ohman, L. P. Keegan, M. A. O'Connell, The RNA-editing enzyme ADAR1 controls innate immune responses to RNA. Cell Rep. 9, 1482-1494 (2014).
37. K. Pestal, C. C. Funk, J. M. Snyder, N. D. Price, P. M. Treuting, D. B. Stetson, Isoforms of RNA-Editing Enzyme ADAR1 Independently Control Nucleic Acid Sensor MDA5-Driven Autoimmunity and Multi-organ Development. Immunity. 43, 933-944 (2015).
38. B. J. Liddicoat, R. Piskol, A. M. Chalk, G. Ramaswami, M. Higuchi, J. C. Hartner, J. B. Li, P. H. Seeburg, C. R. Walkley, RNA editing by ADAR1 prevents MDA5 sensing of endogenous dsRNA as nonself. Science (80-.). (2015), doi:10.1126/science.aac7049.
39. C. K. Pfaller, R. C. Donohue, S. Nersisyan, L. Brodsky, R. Cattaneo, Extensive editing of cellular and viral double-stranded RNA structures accounts for innate immunity suppression and the proviral activity of ADAR1 p 150. PLoS Biol. 16 (2018), doi:10.1371/journal.pbio.2006577.
40. C. X. George, G. Ramaswami, J. B. Li, C. E. Samuel, Editing of Cellular Self-RNAs by Adenosine Deaminase ADAR1 Suppresses Innate Immune Stress Responses. J Biol Chem. 291, 6158-6168 (2016).
41. R. de Reuver, E. Dierick, B. Wiernicki, K. Staes, L. Seys, E. De Meester, T. Muyldermans, A. Botzki, B. N. Lambrecht, F. Van Nieuwerburgh, P. Vandenabeele, J. Maelfait, ADAR1 interaction with Z-RNA promotes editing of endogenous double-stranded RNA and prevents MDA5-dependent immune activation. Cell Rep. 36 (2021), doi:10.1016/j.celrep.2021.109500.
42. E. Eisenberg, E. Y. Levanon, A-to-I RNA editing—Immune protector and transcriptome diversifier. Nat. Rev. Genet. 19 (2018), doi:10.1038/s41576-018-0006-1.
43. T. Nakahama, Y. Kato, T. Shibuya, M. Inoue, J. I. Kim, T. Vongpipatana, H. Todo, Y. Xing, Y. Kawahara, Mutations in the adenosine deaminase ADAR1 that prevent endogenous Z-RNA binding induce Aicardi-Goutières-syndrome-like encephalopathy. Immunity. 54 (2021), doi:10.1016/j.immuni.2021.08.022.
44. X. Guo, R. A. Steinman, Y. Sheng, G. Cao, C. A. Wiley, Q. Wang, An AGS-associated mutation in ADAR1 catalytic domain results in early-onset and MDA5-dependent encephalopathy with IFN pathway activation in the brain. J. Neuroinflammation. 19 (2022), doi:10.1186/s12974-022-02646-0.
45. R. Behrendt, A. Roers, Mouse models for Aicardi-Goutieres syndrome provide clues to the molecular pathogenesis of systemic autoimmunity. Clin Exp Immunol. 175, 9-16 (2014).
46. M. Maurano, J. M. Snyder, C. Connelly, J. Henao-Mejia, C. Sidrauski, D. B. Stetson, Protein kinase R and the integrated stress response drive immunopathology caused by mutations in the RNA deaminase ADAR1. Immunity (2021), doi:10.1016/j.immuni.2021.07.001.
47. H. Jiao, L. Wachsmuth, S. Wolf, J. Lohmann, M. Nagata, G. G. Kaya, N. Oikonomou, V. Kondylis, M. Rogg, M. Diebold, S. E. Tröder, B. Zevnik, M. Prinz, C. Schell, G. R. Young, G. Kassiotis, M. Pasparakis, ADAR1 averts fatal type I interferon induction by ZBP 1. Nature. 607, 776-783 (2022).
48. Y. Pinto, H. Y. Cohen, E. Y. Levanon, Mammalian conserved ADAR targets comprise only a small fragment of the human editosome. Genome Biol. 15, R5 (2014).
49. M. H. Tan, Q. Li, R. Shanmugam, R. Piskol, J. Kohler, A. N. Young, K. I. Liu, R. Zhang, G. Ramaswami, K. Ariyoshi, A. Gupte, L. P. Keegan, C. X. George, A. Ramu, N. Huang, E. A. Pollina, D. S. Leeman, A. Rustighi, Y. P. S. Goh, G. Te. Consortium, D. A. Laboratory, G. Coordinating Center-Analysis Working, G. Statistical Methods groups-Analysis Working, Gte. groups Enhancing, N. I. H. C. Fund, Nih/Nci, Nih/Nhgri, Nih/Nimh, Nih/Nida, N. Biospecimen Collection Source Site, R. Biospecimen Collection Source Site, V. Biospecimen Core Resource, B. Brain Bank Repository-University of Miami Brain Endowment, M. Leidos Biomedical-Project, E. Study, I. Genome Browser Data, E. B. I. Visualization, I. Genome Browser Data, U. of C. S. C. Visualization-Ucsc Genomics Institute, A. Chawla, G. Del Sal, G. Peltz, A. Brunet, D. F. Conrad, C. E. Samuel, M. A. O'Connell, C. R. Walkley, K. Nishikura, J. B. Li, Dynamic landscape and regulation of RNA editing in mammals. Nature. 550, 249-254 (2017).
50. C. J. Herrmann, R. Schmidt, A. Kanitz, P. Artimo, A. J. Gruber, M. Zavolan, PolyASite 2.0: A consolidated atlas of polyadenylation sites from 3′ end sequencing. Nucleic Acids Res. 48 (2020), doi:10.1093/nar/gkz918.
51. C. Mayr, What are 3′ utrs doing? Cold Spring Harb. Perspect. Biol. (2019), doi:10.1101/cshperspect.a034728.
52. B. Bae, P. Miura, Emerging roles for 3′ UTRs in neurons. Int. J. Mol. Sci. 21 (2020), doi:10.3390/ijms21103413.
53. X. Wu, D. P. Bartel, Widespread Influence of 3′-End Structures on Mammalian mRNA Processing and Stability. Cell (2017), doi:10.1016/j.cell.2017.04.036.
54. G. Tushev, C. Glock, M. Heumuller, A. Biever, M. Jovanovic, E. M. Schuman, Alternative 3′ UTRs Modify the Localization, Regulatory Potential, Stability, and Plasticity of mRNAs in Neuronal Compartments. Neuron. 98, 495-511 e6 (2018).
55. P. Miura, S. Shenker, C. Andreu-Agullo, J. O. Westholm, E. C. Lai, Widespread and extensive lengthening of 3′ UTRs in the mammalian brain. Genome Res. 23, 812-825 (2013).
56. L. Li, K. L. Huang, Y. Gao, Y. Cui, G. Wang, N. D. Elrod, Y. Li, Y. E. Chen, P. Ji, F. Peng, W. K. Russell, E. J. Wagner, W. Li, An atlas of alternative polyadenylation quantitative trait loci contributing to complex trait and disease heritability. Nat. Genet. 53 (2021), doi:10.1038/s41588-021-00864-5.
57. L. Wei, S. Lee, S. Majumdar, B. Zhang, P. Sanfilippo, B. Joseph, P. Miura, M. Soller, E. C. Lai, Overlapping Activities of ELAV/Hu Family RNA Binding Proteins Specify the Extended Neuronal 3′ UTR Landscape in Drosophila. Mol. Cell. 80 (2020), doi:10.1016/j.molcel.2020.09.007.
58. H. J. Okano, R. B. Darnell, A hierarchy of Hu RNA binding proteins in developing and adult neurons. J. Neurosci. 17 (1997), doi:10.1523/jneurosci.17-09-03024.1997.
59. W. Akamatsu, H. Fujihara, T. Mitsuhashi, M. Yano, S. Shibata, Y. Hayakawa, H. J. Okano, S. I. Sakakibara, H. Takano, T. Takano, T. Takahashi, T. Noda, H. Okano, The RNA-binding protein HuD regulates neuronal cell identity and maturation. Proc. Natl. Acad. Sci. U.S.A 102 (2005), doi:10.1073/pnas.0407523102.
60. K. D. Mansfield, J. D. Keene, Neuron-specific ELAV/Hu proteins suppress HuR mRNA during neuronal differentiation by alternative polyadenylation. Nucleic Acids Res. 40 (2012), doi:10.1093/nar/gkrl 114.
61. G. Ince-Dunn, H. J. Okano, K. B. Jensen, W. Y. Park, R. Zhong, J. Ule, A. Mele, J. J. Fak, C. W. Yang, C. Zhang, J. Yoo, M. Herre, H. Okano, J. L. Noebels, R. B. Darnell, Neuronal Elav-like (Hu) Proteins Regulate RNA Splicing and Abundance to Control Glutamate Levels and Neuronal Excitability. Neuron. 75 (2012), doi:10.1016/j.neuron.2012.07.009.
62. Y. Kato, T. Iwamori, Y. Ninomiya, T. Kohda, J. Miyashita, M. Sato, Y. Saga, ELAVL2-directed RNA regulatory network drives the formation of quiescent primordial follicles. EMBO Rep. 20 (2019), doi:10.15252/embr.201948251.
63. P. Desai, S. Person, Incorporation of the Green Fluorescent Protein into the Herpes Simplex Virus Type 1 Capsid. J. Virol. 72 (1998), doi:10.1128/jvi.72.9.7563-7568.1998.
64. S. Y. Zhang, N. E. Clark, C. A. Freije, E. Pauwels, A. J. Taggart, S. Okada, H. Mandel, P. Garcia, M. J. Ciancanelli, A. Biran, F. G. Lafaille, M. Tsumura, A. Cobat, J. Luo, S. Volpi, B. Zimmer, S. Sakata, A. Dinis, O. Ohara, E. J. Garcia Reino, K. Dobbs, M. Hasek, S. P. Holloway, K. McCammon, S. A. Hussong, N. DeRosa, C. E. Van Skike, A. Katolik, L. Lorenzo, M. Hyodo, E. Faria, R. Halwani, R. Fukuhara, G. A. Smith, V. Galvan, M. J. Damha, S. Al-Muhsen, Y. Itan, J. D. Boeke, L. D. Notarangelo, L. Studer, M. Kobayashi, L. Diogo, W. G. Fairbrother, L. Abel, B. R. Rosenberg, P. J. Hart, A. Etzioni, J. L. Casanova, Inborn Errors of RNA Lariat Metabolism in Humans with Brainstem Viral Infection. Cell. 172 (2018), doi:10.1016/j.cell.2018.02.019.
65. F. G. Lafaille, O. Harschnitz, Y. S. Lee, P. Zhang, M. L. Hasek, G. Kerner, Y. Itan, O. Ewaleifoh, F. Rapaport, T. M. Carlile, M. E. Carter-Timofte, D. Paquet, K. Dobbs, B. Zimmer, D. Gao, M. F. Rojas-Duran, D. Kwart, V. Rattina, M. J. Ciancanelli, J. L. McAlpine, L. Lorenzo, S. Boucherit, F. Rozenberg, R. Halwani, B. Henry, N. Amenzoui, Z. Alsum, L. Marques, J. A. Church, S. Al-Muhsen, M. Tardieu, A. A. Bousfiha, S. R. Paludan, T. H. Mogensen, L. Quintana-Murci, M. Tessier-Lavigne, G. A. Smith, L. D. Notarangelo, L. Studer, W. Gilbert, L. Abel, J. L. Casanova, S. Y. Zhang, Human SNORA31 variations impair cortical neuron-intrinsic immunity to HSV-1 and underlie herpes simplex encephalitis. Nat. Med. 25 (2019), doi:10.1038/s41591-019-0672-3.
66. S. R. Paludan, T. H. Mogensen, Constitutive and latent immune mechanisms exert ‘silent’ control of virus infections in the central nervous system. Curr. Opin. Immunol. 72 (2021), doi:10.1016/j.coi.2021.05.004.
67. B. Ogunjimi, S. Y. Zhang, K. B. Sorensen, K. A. Skipper, M. Carter-Timofte, G. Kerner, S. Luecke, T. Prabakaran, Y. Cai, J. Meester, E. Bartholomeus, N. A. Bolar, G. Vandeweyer, C. Claes, Y. Sillis, L. Lorenzo, R. A. Fiorenza, S. Boucherit, C. Dielman, S. Heynderickx, G. Elias, A. Kurotova, A. Vander Auwera, L. Verstraete, L. Lagae, H. Verhelst, A. Jansen, J. Ramet, A. Suls, E. Smits, B. Ceulemans, L. Van Laer, G. P. Wilson, J. Kreth, C. Picard, H. Von Bernuth, J. Fluss, S. Chabrier, L. Abel, G. Mortier, S. Fribourg, J. G. Mikkelsen, J. L. Casanova, S. R. Paludan, T. H. Mogensen, Inborn errors in RNA polymerase III underlie severe varicella zoster virus infections. J. Clin. Invest. 127 (2017), doi:10.1172/JC192280.
68. E. G. Conlon, J. L. Manley, RNA-binding proteins in neurodegeneration: mechanisms in aggregate. Genes Dev. 31, 1509-1528 (2017).
69. M. Monje, A. Iwasaki, The neurobiology of long COVID. Neuron. 110, 3484-3496 (2022).
70. J. C. Udeochu, S. Amin, Y. Huang, L. Fan, E. R. S. Torres, G. K. Carling, B. Liu, H. McGurran, G. Coronas-Samano, G. Kauwe, G. A. Mousa, M. Y. Wong, P. Ye, R. K. Nagiri, I. Lo, J. Holtzman, C. Corona, A. Yarahmady, M. T. Gill, R. M. Raju, S. A. Mok, S. Gong, W. Luo, M. Zhao, T. E. Tracy, R. R. Ratan, L. H. Tsai, S. C. Sinha, L. Gan, Tau activation of microglial cGAS-IFN reduces MEF2C-mediated cognitive resilience. Nat. Neurosci. 26 (2023), doi:10.1038/s41593-023-01315-6.
71. C. H. Yu, S. Davidson, C. R. Harapas, J. B. Hilton, M. J. Mlodzianoski, P. Laohamonthonkul, C. Louis, R. R. J. Low, J. Moecking, D. De Nardo, K. R. Balka, D. J. Calleja, F. Moghaddas, E. Ni, C. A. McLean, A. L. Samson, S. Tyebji, C. J. Tonkin, C. R. Bye, B. J. Turner, G. Pepin, M. P. Gantier, K. L. Rogers, K. McArthur, P. J. Crouch, S. L. Masters, TDP-43 Triggers Mitochondrial DNA Release via mPTP to Activate cGAS/STING in ALS. Cell. 183 (2020), doi:10.1016/j.cell.2020.09.020.
72. Q. Li, M. J. Gloudemans, J. M. Geisinger, B. Fan, F. Aguet, T. Sun, G. Ramaswami, Y. I. Li, J. B. Ma, J. K. Pritchard, S. B. Montgomery, J. B. Li, RNA editing underlies genetic risk of common inflammatory diseases. Nature. 608, 569-577 (2022).
73. J. J. Ishizuka, R. T. Manguso, C. K. Cheruiyot, K. Bi, A. Panda, A. Iracheta-Vellve, B. C. Miller, P. P. Du, K. B. Yates, J. Dubrot, I. Buchumenski, D. E. Comstock, F. D. Brown, A. Ayer, I. C. Kohnle, H. W. Pope, M. D. Zimmer, D. R. Sen, S. K. Lane-Reticker, E. J. Robitschek, G. K. Griffin, N. B. Collins, A. H. Long, J. G. Doench, D. Kozono, E. Y. Levanon, W. N. Haining, Loss of ADAR1 in tumours overcomes resistance to immune checkpoint blockade. Nature. 565, 43-48 (2019).
74. P. Mehdipour, S. A. Marhon, I. Ettayebi, A. Chakravarthy, A. Hosseini, Y. Wang, F. A. de Castro, H. Loo Yau, C. Ishak, S. Abelson, C. A. O'Brien, D. D. De Carvalho, Epigenetic therapy induces transcription of inverted SINEs and ADAR1 dependency. Nature. 588 (2020), doi:10.1038/s41586-020-2844-1.
75. T. Zhang, C. Yin, A. Fedorov, L. Qiao, H. Bao, N. Beknazarov, S. Wang, A. Gautam, R. M. Williams, J. C. Crawford, S. Peri, V. Studitsky, A. A. Beg, P. G. Thomas, C. Walkley, Y. Xu, M. Poptsova, A. Herbert, S. Balachandran, ADAR1 masks the cancer immunotherapeutic promise of ZBP1-driven necroptosis. Nature. 606, 594-602 (2022).
76. B. P. Daniels, R. S. Klein, Knocking on Closed Doors: Host Interferons Dynamically Regulate Blood-Brain Barrier Function during Viral Infections of the Central Nervous System. PLoS Pathog. 11 (2015), doi:10.1371/journal.ppat.1005096.
77. T. Blank, M. Prinz, Type I interferon pathway in CNS homeostasis and neurological disorders. Glia. 65 (2017), doi:10.1002/glia.23154.
78. M. D. Robinson, D. J. McCarthy, G. K. Smyth, edgeR: a Bioconductor package for differential expression analysis of digital gene expression data. Bioinformatics. 26, 139-140 (2010).
79. G. Yu, L. G. Wang, Y. Han, Q. Y. He, ClusterProfiler: An R package for comparing biological themes among gene clusters. Omi. A J. Integr. Biol. 16 (2012), doi:10.1089/omi.2011.0118.

1-16. (canceled)

17. A method of treating a neurodegenerative disease or neuroinflammatory condition in a subject comprising administering a HuB-effector agent to the subject which decreases expression of, and/or inhibits activity of, HuB in the subject, thereby treating the neurodegenerative disease or neuroinflammatory condition.

18. The method of claim 17, wherein the HuB-effector agent is delivered to a brain or central nervous system of the subject.

19. The method of claim 17, wherein the HuB-effector agent is delivered to neurons, preferably differentiated neurons, preferably post-mitotic neurons, of the subject.

20. The method of claim 17, wherein the decrease in the expression of, or inhibition of the activity of, HuB occurs in neurons of the subject, preferably differentiated neurons, preferably post-mitotic neurons.

21. The method of claim 17, wherein the HuB-effector agent comprises a small interfering RNA (siRNA), a short hairpin RNA (shRNA), a CRISPR RNA (crRNA), a single-guide RNA (sgRNA), and/or a prime editing guide RNA (pegRNA) which has complementarity to a nucleotide molecule encoding HuB, preferably a gene or transcript which comprises at least one portion encoding HuB.

22. The method of claim 17, wherein the HuB-effector agent comprises an antibody, or portion thereof, which binds HuB, preferably wherein the antibody inhibits the activity of HuB, preferably RNA-binding activity of HuB.

23. The method of claim 17, wherein the HuB-effector agent is delivered to the subject via adeno-associated virus (AAV)-mediated delivery, lentiviral delivery, a virus-like particle (VLP), or Sindbis viral delivery.

24. A method of reducing immunostimulatory double-stranded RNA (dsRNA) levels and/or type I interferon (IFN) expression in a neuron, the method comprising delivering a HuB-effector agent to the neuron which decreases the expression of, and/or inhibits activity of, HuB in the neuron, thereby reducing immunostimulatory double-stranded RNA (dsRNA) levels and/or type I interferon (IFN) expression in the neuron.

25. The method of claim 24, wherein an average 3′UTR length of transcripts expressed by the neuron is decreased, preferably wherein the neuron displays a decreased average Percentage of Distal poly-A sites Usage Index (PDUI) value relative to a non-treated neuron.

26. The method of claim 24, wherein MDA5-mediated type I interferon production by the neuron is decreased.

27. A method of reducing immunostimulatory double-stranded RNA (dsRNA) levels and/or type I interferon (IFN) expression in a neuron, the method comprising delivering a HuC-effector agent to the neuron which decreases the expression of, and/or inhibits the activity of, HuC in the neuron, thereby reducing immunostimulatory double-stranded RNA (dsRNA) levels and/or type I interferon (IFN) expression in the neuron.

28. The method of claim 27, wherein the average 3′UTR length of transcripts expressed by the neuron is decreased, preferably wherein the neuron displays a decreased average Percentage of Distal poly-A sites Usage Index (PDUI) value relative to a non-treated neuron.

29. The method of claim 27, wherein MDA5-mediated type I interferon production by the neuron is decreased.

30. A method of treating or preventing a viral infection or cancer in a subject comprising administering an agent to the subject which increases the presence and/or activity of, HuB, HuC, and HuD in the subject.

31. The method of claim 30, comprising administering to the subject HuB, HuC, and/or HuD proteins, or variants thereof, or administering to the subject one or more nucleic acid molecules that express HuB, HuC, and/or HuD, or variants thereof.

32. The method of claim 31, wherein the HuB, HuC, and/or HuD proteins and/or one or more nucleic acid molecules are delivered to the subject via adeno-associated virus (AAV)-mediated delivery, lentiviral delivery, a virus-like particle (VLP), or Sindbis viral delivery.

33. The method of claim 30, wherein the subject is susceptible to infection with a herpes simplex virus, Zika virus, Sindbis virus, and/or a flavirius.
